(19) United States
(12) Patent Application Publication
(10) Pub. No.: US 2023/0332148 A1
(43) Pub. Date: Oct. 19, 2023

(54) DISCONTINUOUS OLIGONUCLEOTIDE LIGANDS

(71) Applicant: Rheinische Friedrich-Wilhelms-Universitat Bonn, Bonn (DE)

(72) Inventors: Marion Goldeck, Bonn (DE); Janos Ludwig, Gottingen (DE)

(21) Appl. No.: 18/047,487

(22) Filed: Oct. 18, 2022

Related U.S. Application Data
(63) Continuation of application No. 15/741,193, filed on Dec. 29, 2017, now abandoned.

(30) Foreign Application Priority Data
Jul. 2, 2015 (DE) 102015008536.0

(51) Int. Cl. C12N 15/113; C12N 15/11; C12N 15/117; A61K 31/713

(52) U.S. Cl. CPC C12N 15/113; A61K 31/713; C12N 15/111; C12N 15/117; C12N 2310/14; C12N 2310/317; C12N 2310/321; C12N 2310/3519; C12N 2310/51

(57) ABSTRACT

The present invention relates to an oligonucleotide conjugate K of the structure RNA1-B-RNA2 RNA3 RNA4 or a pharmaceutically active salt thereof, wherein each RNA1, RNA2, RNA3 and RNA4 is a strand of a ribonucleic acid or of an analogue or of a derivative thereof, wherein B is a divalent linker that covalently bonds the 5′ terminus of RNA1 to the 5′ terminus of RNA2 or the 3′ terminus of RNA1 to the 3′ terminus of RNA2, and wherein RNA3 and RNA4 are not covalently bonded to each other. The invention further relates to the medical and non-medical use of such an oligonucleotide conjugate K and to corresponding manufacturing methods.

This application includes a sequence listing submitted electronically by the file name 52710A_Seqlisting.XML; Size: 88,886 bytes; Created: Sep. 30, 2022, and is incorporated herein by reference.

The present invention relates to an oligonucleotide conjugate K of structure

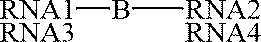

or a pharmaceutically acceptable salt thereof, wherein RNA1, RNA2, RNA3 and RNA4 are each strands of a ribonucleic acid or an analog or derivative thereof, wherein B represents a bivalent linker that covalently bonds the 5′ terminus of RNA1 to the 5′ terminus of RNA2 or the 3′ terminus of RNA1 to the 3′ terminus of RNA2, and wherein RNA3 and RNA4 are not covalently bonded to one another. The present invention further relates to the medical and non-medical use of such an oligonucleotide conjugate K and to production processes therefor.

Some cytosolic proteins involved in protective mechanisms for the combating of infections, for instance cytosolic helicases, can be activated by particular double-stranded ribonucleic acids (dsRNAs). The activation of such cytosolic proteins and initiation of the corresponding protective mechanisms can serve for treatment of neoplasias, infections and/or uncontrolled immune responses.

For instance, the cytosolic helicase RIG-I (retinoic acid-inducible gene I) can be activated by dsRNAs (Yoneyama et al., 2004, nat. Immunol. 5(7):730-737; Marques et al., 2006, Nat. Biotech. 24(5):559-565). Those RNAs having 5′-terminal triphosphate have been found to be even more effective (cf. Hornung et al., 2006, Science 314:994-997; Pichelmair et al., 2006, Science 314:997-1001; Schmidt et al., 2009, PNAS 106(29):12067-12072; Schlee et al., 2009, Immunity 31:25-34). However, the stability and bioactivity of these molecules is still inadequate and in need of improvement.

In other contexts, there have been descriptions of ribonucleic acid (RNA) molecules, some with elevated stability, in which, for instance, two individual strands are each linked via their 3′ termini (3′-3′ linkage) (cf. US 2005/0026861, US 6,489,464, US 2008/0171712). These also seem to have certain advantageous properties. However, these cannot be used directly for activation of cytosolic proteins that trigger protective mechanisms for the combating of neoplasias, injections and/or uncontrolled immune response, for instance RIG-I. These are generally single strands, refolded strands in which portions of the two covalently bonded RNA strands hybridize with one another, RNAs hybridized to a common strand or polymeric structures composed of 3′-3′-linked structures. However, what is not known is hybridization of each of the two strands that are joined to one another via a 5′-5′ linkage or a 3′-3′ linkage to a complementary single, unbound RNA strand.

None of the structures known from the prior art enables activation of cellular factors, for instance RIG-I, to the desired degree and is simultaneously easily obtainable synthetically.

It was therefore desirable to provide oligonucleotide conjugates having improved properties for activation of cellular factors, for instance RIG-I, which are additionally also easily obtainable synthetically.

It has been found that, surprisingly, an oligonucleotide conjugate K in which at least two RNA strands are 5′-5′- or 3′-3′-linked and are at least partly hybridized with further single RNA strands have particularly advantageous properties in relation to the activation of cytosolic proteins involved in protective mechanisms for the combating of neoplasias, injections and/or uncontrolled immune response, particularly cytosolic helicases, especially RIG-I.

In a first aspect, the present invention relates to an oligonucleotide conjugate K of the structure

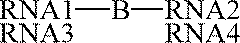

or a pharmaceutically compatible salt thereof, wherein:
RNA 1 represents a first strand of a ribonucleic acid or an analog or derivative thereof of at least six nucleotides in length;
RNA2 represents a second strand of a ribonucleic acid or an analog or derivative thereof of at least six nucleotides in length;
RNA3 represents a third strand of a ribonucleic acid or an analog or derivative thereof of at least six nucleotides in length, which forms at least five complementary base pairs with RNA1;
RNA4 represents a fourth strand of a ribonucleic acid or an analog or derivative thereof of at least six nucleotides in length, which forms at least five complementary base pairs with RNA2; and
B represents a bivalent linker that covalently bonds the 5′ terminus of RNA1 to the 5′ terminus of RNA2 or the 3′ terminus of RNA1 to the 3′ terminus of RNA2,
wherein RNA3 and RNA4 are not covalently bonded to one another.

In other words, the bivalent linker B joins RNA1 and RNA2 to one another so as to give a 5′-5′ linkage or a further 3′-3′ linkage in the oligonucleotide conjugate K of the invention. Preferably, there is just a single 5′-5′ linkage or 3′-3′ linkage in the entire oligonucleotide conjugate K.

Preferably, the oligonucleotide conjugate K or a pharmaceutically compatible salt thereof consists of the structure

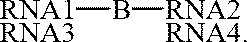

It will be apparent to the person skilled in the art that a ribonucleic acid, an analog or derivative thereof also includes the respective pharmaceutically compatible salts thereof that form or can form, depending on the pH, in an aqueous environment.

Examples of countercations are, for instance, H⁺, Na⁺, K⁺, Mg²⁺, Ca²⁺, Zn²+ and the cations of amino acids (e.g. lysine, arginine, proline) and choline.

RNA1, RNA2, RNA3 and RNA4 each represent a continuous coherent strand. RNA1, RNA2, RNA3 and RNA4 therefore each have a 3′ terminus and a 5′ terminus. Therefore, there is no interruption of the sequence in RNA1, RNA2, RNA3 and RNA4 to the effect that a further 5′-5′ linkage or a further 3′-3′ linkage occurs within these strands.

Expressed in more detail, one of the following structures is present:

Oligonucleotide conjugate K of the invention with 5′-5′ linkage:

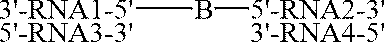

Oligonucleotide conjugate K of the invention with 3′-3′ linkage:

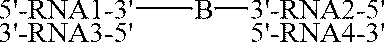

An analog of a ribonucleic acid (RNA analog) may be any RNA analog known in the prior art. For example, an RNA analog may be selected from the group consisting of peptide nucleic acid (PNA), morpholinos, glycol nucleic acid (GNA), threose nucleic acid (TNA), locked nucleosides (LNA) and 2′,3′-seco-nucleosides (UNA). In addition, the oligonucleotides may include non-nucleotide units and/or side chain modifications. For example, the 2′—OH of a ribonucleoside subunit may be replaced by a group selected from OR, R, halogen, SH, SR, NH₂, NHR, NR₂ and CN, where R is an optionally substituted C₁-₆-alkyl, C₂-₆-alkenyl, C₂-₆-alkynyl and halogen is preferably F, CI, Br or I. In addition, the ribose may be replaced by another sugar, for example another pentose (e.g. arabinose). Linkage by two sugars is also possible via a 2′-fluoroarabinonucleoside subunit. It is likewise possible to use phosphorothioates, phosphorodithioates, methylphosphonates, phosphoramidates, boranophosphates or other modified bonds.

A derivative of a ribonucleic acid (RNA derivative) may be any modified, chemically or biologically altered form of a ribonucleic acid. For example, an RNA derivative may be methylated RNA. For example, one base or two or more bases or all bases may be alkylated (e.g. methylated) and/or halogenated (e.g. fluorinated). It is also possible for one or more of the sugar residues (ribose residues) of the nucleic acid to be modified once or more than once. For example, a ribose residue may be alkylated (e.g. methylated). Preferably, a ribose residue may be 2′—O—alkylated (e.g. 2′—O—methylated). Alternatively or additionally, one or more phosphothioates may be present as bonds between the nucleotides.

It is also possible to use non-standard nucleobases, for example 5-(2-amino)-propyluracil, 5-bromouracil, hypoxanthine; 2,6-diaminopurine; 8-bromoguanine, 7-deazaguanine, 7-deazaadenine; N⁶-methyladenine, N⁶-dimethyladenine or 5-nitroindole.

Further modifications usable in connection with the present invention are shown in WO 2014/049079. Examples of RNA derivatives are shown in the examples section of the present application.

Preferably, the activity of the correspondingly modified ribonucleic acid is not too significantly restricted. For instance, preferably at least 60%, even more preferably at least 70%, even more preferably at least 80%, especially preferably at least 90%, of the activity (for example of the RIG-I-activating activity) is conserved compared to the corresponding unmodified RNA. It is even possible for there to be a rise in activity, which is particularly preferred.

Preferably, the modifications increase the biological stability and storage stability of the nucleic acid strand and/or the entire oligonucleotide conjugate K according to the present invention.

It will be apparent to the person skilled in the art that not necessarily all nucleotides in a strand are homogeneously all ribonucleic acid nucleotides, RNA analog nucleotides or RNA derivative nucleotides. Instead, it is also possible for single nucleotides to be RNA analog nucleotides and/or RNA derivative nucleotides and for the rest of the strand to consist (largely) of ribonucleic acid nucleotides, or vice versa.

The oligonucleotide conjugate K of the invention may address a particular target.

In a preferred embodiment, the oligonucleotide conjugate K is an activator of cytosolic helicase retinoic acid-inducible gene I (RIG-I).

Preferably, RIG-I as used herein has at least 80% homology to gene name DDX58 or UniProt ID O95786 or OMIM ID 609631. It is a helicase. RIG-I can also be referred to as probable ATP-dependent RNA helicase (DDX58). More preferably, RIG-I as used herein has at least 90%, even more preferably at least 95%, even more preferably at least 98%, homology, especially identity, to gene name DDX58.

Cytosolic helicase retinoic acid-inducible gene I (RIG-I) is a cytosolic helicase which serves as nucleic acid receptor in the congenital immune system. The activation of RIG-I typically leads to triggering of a signal cascade which leads to excretion of numerous cytokines and chemokines, especially of type I interferons (e.g. interferon alpha (INFa)) and promotes congenital and adaptive immune responses.

An end structure in the dimer unit RNA1/RNA3 and/or the dimer unit RNA2/RNA4 may be complementary to a domain of the target protein, for instance to a C-terminal domain, for example to the C-terminal domain (CTD) of the RIG-I protein. This may especially be the case when the 5′ end of the RNA1/RNA3 dimer unit and/or of the RNA2/RNA4 dimer unit at the opposite end from the linker B forms a double strand with a blunt end. The oligonucleotide conjugates K described here, in a particularly preferred embodiment, are characterized in that they have two identical CTD-binding strands without overhangs (blunt ends) in symmetric arrangement, which can be advantageous for the interaction with RIG-I.

Preferably, an activator of RIG-I is characterized in that the secretion of interferon alpha (IFNα) by the cells that are treated with an oligonucleotide conjugate K of the invention is higher than in the case of corresponding cells that are not treated with an oligonucleotide conjugate K of the invention. The cells here are preferably peripheral blood mononuclear cells (PBMCs), which have especially preferably been pretreated with standard doses of chloroquine.

More preferably, in the case of addition of 5 nM of the oligonucleotide conjugate K of the invention, IFNα secretion from chloroquine-treated PBMCs rises by at least 50%, preferably by at least double, more preferably by at least five times, especially at least 10 times or even more than 20 times compared to corresponding chloroquine-treated PBMCs without addition of the oligonucleotide conjugate K of the invention. This is also further illustrated by the examples shown below.

The sequences RNA1 and RNA2 may be different, may be similar to one another or may be identical to one another. For instance, the sequences of RNA1 and RNA2 may have a sequence homology of less than 60%, of at least 60%, of at least 70%, of at least 80%, of at least 90%, of at least 95%, of at least 98% or of 100% (i.e. complete identity). Preferably, RNA1 and RNA2 are at least similar to one another, and more preferably have at least 60%, even more preferably at least 70%, even more preferably at least 80%, even more preferably at least 90%, even more preferably at least 95%, even more preferably at least 98% and especially more than 98% sequence homology.

In an especially preferred embodiment, the sequence of RNA2 is identical to the sequence of RNA 1 .

It is likewise possible for the sequences RNA3 and RNA4 to be different, for them to be similar to one another or for them to be identical. For instance, the sequences of RNA3 and RNA4 may have a sequence homology of less than 60%, of at least 60%, of at least 70%, of at least 80%, of at least 90%, of at least 95%, of at least 98% or of 100% (i.e. complete identity). Preferably, RNA3 and RNA4 are at least similar to one another and more preferably have at least 60%, even more preferably at least 70%, even more preferably at least 80%, even more preferably at least 90%, even more preferably at least 95%, even more preferably at least 98% and especially more than 98% sequence homology.

In a particularly preferred embodiment, the sequence of RNA3 is identical to the sequence of RNA4.

As apparent from the structure shown above and as defined in the text, RNA3 is at least partly complementary to RNA1, and RNA4 to RNA2. Therefore, even more preferably, RNA1 is at least similar to RNA2 and RNA3 to RNA4, and they more preferably have at least 60%, even more preferably at least 70%, even more preferably at least 80%, even more preferably at least 90%, even more preferably at least 95%, even more preferably at least 98% and especially more than 98% sequence homology to one another.

Most preferably, the sequence of RNA2 is identical to the sequence of RNA1 and the sequence of RNA3 is identical to the sequence of RNA4.

In this case, the oligonucleotide conjugate K is (largely) symmetrical. As shown above, B in the structure shown above represents a bivalent linker that covalently bonds the 5′ terminus of RNA1 to the 5′ terminus of RNA2 or the 3′ terminus of RNA1 to the 3′ terminus of RNA2. Preferably, B represents a bivalent linker that covalently bonds the 5′ terminus of RNA1 to the 5′ terminus of RNA2. Therefore, RNA1 and RNA2 are preferably each bonded covalently to the bivalent linker B via their 5′ termini.

The 5′ termini of RNA3 and RNA4 may be triphosphate residues, triphosphate analog residues, free hydroxyl groups or other residues, bound to solid supports or polymeric structures. Preferably, the 5′ termini of RNA3 and RNA4 each have triphosphate residues, triphosphate analog residues or free hydroxyl groups. More preferably, the 5′ termini of RNA3 and RNA4 each have triphosphate residues or free hydroxyl groups. Even more preferably still, the 5′ termini of RNA3 and RNA4 each have free hydroxyl groups.

In a more preferred embodiment, B represents a bivalent linker that covalently bonds the 5′ terminus of RNA1 to the 5′ terminus of RNA2, and RNA3 and RNA4 each have triphosphate residues, triphosphate analog residues or free hydroxyl groups at their 5′ termini.

In an even more preferred embodiment, B represents a bivalent linker that covalently bonds the 5′ terminus of RNA1 to the 5′ terminus of RNA2, and RNA3 and RNA4 each have triphosphate residues or free hydroxyl groups at their 5′ termini.

In a yet more preferred embodiment, B represents a bivalent linker that covalently bonds the 5′ terminus of RNA1 to the 5′ terminus of RNA2, and RNA3 and RNA4 each have triphosphate residues at their 5′ termini.

This most preferably results in the following structure for the oligonucleotide conjugate K with 5′-5′ linkage and triphosphate residues (ppp):

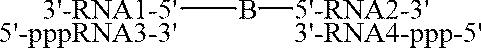

Alternatively, the 3′ termini of RNA1 and RNA2 may each be bonded to the bivalent linker B. In that case, the 5′ termini of RNA1 and RNA2 may have triphosphate residues, triphosphate analog residues, free hydroxyl groups or other residues, bound to solid supports or polymeric structures.

In a preferred embodiment, B in that case represents a bivalent linker residue that covalently bonds the 3′ terminus of RNA1 to the 3′ terminus of RNA2 and RNA1 and RNA2 each have, at their 5′ termini, triphosphate residues, triphosphate analog residues or free hydroxyl groups.

When the bivalent linker residue is phosphate, the first 3′-terminal nucleotide is preferably substituted (e.g. 2′-substituted (e.g. 2′-methylated)). This may increase its chemical stability.

In a more preferred embodiment, B in that case represents a bivalent linker residue that covalently bonds the 3′ terminus of RNA1 to the 3′ terminus of RNA2, and RNA1 and RNA2 each have, at their 5′ termini, triphosphate residues or free hydroxyl groups.

In an even more preferred embodiment, B in that case represents a bivalent linker residue that covalently bonds the 3′ terminus of RNA1 to the 3′ terminus of RNA2, and RNA1 and RNA2 each have, at their 5′ termini, triphosphate residues.

As set out above, the sequences RNA1, RNA2, RNA3 and RNA4 each have a length of at least six nucleotides.

In a preferred embodiment, RNA1, RNA2, RNA3 and RNA4 each have a length of between 10 and 50, preferably between 15 and 40, more preferably between 19 and 30 and especially between 20 and 25 nucleotides.

For example, the length of nucleotides may in each case independently be 19, 20, 21, 22, 23, 24, 25, 26, 27, 28 or 29 nucleotides.

Preferably, the oligonucleotide conjugate K of the invention has double strands each of at least 19 base pairs (bp) in length, formed by RNA1/RNA3 and RNA2/RNA4. For example, the double strands formed by RNA1/RNA3 and RNA2/RNA4 may each independently, for example, have a length of 19, 20, 21, 22, 23, 24, 25, 26, 27, 28 or 29 bp.

In the oligonucleotide conjugate K, RNA1 and RNA3, and RNA2 and RNA4 may each have an overhang of the 5′-terminal nucleotide residues or may not have an overhang of the 5′-terminal nucleotide residues. Preferably, RNA1 and RNA3, and RNA2 and RNA4 each do not have an overhang of the 5′-terminal nucleotide residues.

In addition, in the oligonucleotide conjugate K, RNA1 and RNA3, and RNA2 and RNA4 may each have an overhang of the 3′-terminal nucleotide residues or not have an overhang of the 3′-terminal nucleotide residues. Preferably, RNA1 and RNA3, and RNA2 and RNA4 each have an overhang of not more than five, preferably not more than four, more preferably not more than three and even more preferably not more than two nucleotides, yet more preferably not more than one nucleotide, of the 5′-terminal nucleotide residues. Especially preferably, RNA1 and RNA3, and RNA2 and RNA4 each do not have any overhang of the 5′-terminal nucleotide residues.

Therefore, in a preferred embodiment, RNA1 and RNA3, and RNA2 and RNA4 each do not have an overhang of the 5′-terminal nucleotide residues and an overhang of not more than five, preferably not more than four, more preferably not more than three and even more preferably not more than two nucleotides, even more preferably not more than one nucleotide, of the 3′-terminal nucleotide residues.

In an even more preferred embodiment, RNA1 and RNA3, and RNA2 and RNA4 each do not have any overhang of the 5′-terminal nucleotide residues and do not have a difference in the length of the nucleotide strand.

In a very particularly preferred embodiment, RNA1 is fully complementary to RNA3 and RNA2 is fully complementary to RNA4.

Therefore, in that case, RNA1 has the same length as RNA3 and RNA2 has the same length as RNA4, and all bases preferably enter into corresponding base pairs. In that case, there is neither a 5′ overhang nor a 3′ overhang, but rather a blunt end.

Since, as set out above, RNA1 and RNA2 are preferably at least similar, especially (largely) identical, it is especially preferable that the strands RNA1, RNA2, RNA3, RNA4 are each of equal length.

In a particularly preferred embodiment, therefore, the sequence of RNA2 is identical to the sequence of RNA1 and the sequence of RNA3 is identical to the sequence of RNA4,
RNA 1 is fully complementary to RNA3,
RNA2 is fully complementary to RNA4, and
the strands RNA1, RNA2, RNA3, RNA4 are each of equal length.

The bivalent linker B in the oligonucleotide conjugate K of the invention may be of any chemical nature, provided that RNA1 and RNA2 are covalently bonded to one another. Preferably, the bivalent linker B has a molecular weight of not more than 1500 Da.

In a preferred embodiment, the bivalent linker B is selected from the group consisting of a phosphodiester linker or an analog or derivative thereof, a glycol linker, a polyethylene glycol linker, a carbohydrate linker, a ribonucleic acid linker, a deoxyribonucleic acid linker, an unsubstituted or substituted C₁-C₁₂-alkyl linker, an amino acid linker, a peptide linker, and combinations of two or more of these, wherein the linker has a molecular weight of not more than 1500 Da.

More preferably, the bivalent linker B has a molecular weight of not more than 1000 Da auf, especially a molecular weight of not more than 500 Da. Examples of bivalent linkers B are illustrated in the examples section below.

In a particularly preferred embodiment, the bivalent linker B is a phosphodiester linker:

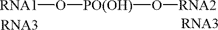

or a pharmaceutically compatible salt thereof:

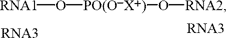

where X⁺ is a pharmaceutically acceptable cation, for example H⁺, Na⁺, K⁺, Ca²⁺, Mg²⁺ etc.

RNA1, RNA2, RNA3 and RNA4 may each independently have any desired sequence.

Preferably, at least one of the strands selected from RNA1, RNA2, RNA3 and RNA4 has a sequence homology to SEQ ID NO: 1 or SEQ ID NO: 2 of at least 80%. These sequences are preferably single strands and form barely any, if any, intramolecular tertiary structures, which facilitates later hybridization with the respective complementary strands. The sequences are shown below. More preferably, at least two of the strands selected from RNA1, RNA2, RNA3 and RNA4 have a sequence homology to SEQ ID NO: 1 or SEQ ID NO: 2 of at least 80%. Even more preferably, two of the strands selected from RNA1, RNA2, RNA3 and RNA4 have a sequence homology to SEQ ID NO: 1 of at least 80% and the two others have a sequence homology to SEQ ID NO: 2 of at least 80%. In this context, RNA1 and RNA3 do not have the same sequences, but preferably the respective other sequence, and RNA2 and RNA4 do not have the same sequences, but preferably the respective other sequence.

In a more preferred embodiment, RNA1 or RNA3 have a sequence homology to SEQ ID NO: 1 of at least 80%.

In a more preferred embodiment, RNA1 has a sequence homology to SEQ ID NO: 1 of at least 80%:

5'-GACGCUGACCCUGAAGUUCAUCUU-3' (SEQ ID NO: 1)

More preferably, RNA1 has a sequence homology to SEQ ID NO: 1 of at least 90%, even more preferably of at least 95%, even more preferably of at least 98%. More particularly, RNA1 has SEQ ID NO: 1.

In an even more preferred embodiment, RNA1 and RNA2 or RNA3 and RNA4 each have a sequence homology to SEQ ID NO: 1 of at least 80%.

In an even more preferred embodiment, RNA1 and RNA2 each have a sequence homology to SEQ ID NO: 1 of at least 80%.

More preferably, RNA1 and RNA2 or RNA3 and RNA4 each have a sequence homology to SEQ ID NO: 1 of at least 90%, even more preferably of at least 95%, even more preferably of at least 98%. More particularly, RNA1 and RNA2 each have SEQ ID NO: 1.

Even more preferably, RNA1 and RNA2 each have a sequence homology to SEQ ID NO: 1 of at least 90%, even more preferably of at least 95%, even more preferably of at least 98%. More particularly, RNA1 and RNA2 each have SEQ ID NO: 1.

Alternatively, RNA1 and/or RNA2 may also have a sequence homology to SEQ ID NO: 2 of at least 80%, preferably of at least 90%, even more preferably of at least 95%, even more preferably of at least 98%.

Preferably, RNA3 has a sequence homology to SEQ ID NO: 2 of at least 80%:

5'-AAGAUGAACUUCAGGGUCAGCGUC-3' (SEQ ID NO: 2)

More preferably, RNA3 has a sequence homology to SEQ ID NO: 2 of at least 90%, even more preferably of at least 95%, even more preferably of at least 98%. More particularly, RNA3 has SEQ ID NO: 2.

Even more preferably, RNA3 and RNA4 each have a sequence homology to SEQ ID NO: 2 of at least 80%. Even more preferably, RNA3 and RNA4 each have a sequence homology to SEQ ID NO: 2 of at least 90%, even more preferably of at least 95%, even more preferably of at least 98%. More particularly, RNA3 and RNA4 each have SEQ ID NO: 2.

Alternatively, RNA3 and/or RNA4 may also have a sequence homology to SEQ ID NO: 1 of at least 80%, preferably of at least 90%, even more preferably of at least 95%, even more preferably of at least 98%.

In a particularly preferred embodiment, RNA1 and RNA2 or RNA3 and RNA4 each have a sequence homology to SEQ ID NO: 1 of at least 80%, and RNA3 and RNA4 or RNA1 and RNA2 each have a sequence homology to SEQ ID NO: 2 of at least 80%.

In a very particularly preferred embodiment, RNA1 and RNA2 each have a sequence homology to SEQ ID NO: 1 of at least 80% and RNA3 and RNA4 each have a sequence homology to SEQ ID NO: 2 of at least 80%.

Alternatively, RNA1 and RNA2 may each have a sequence homology to SEQ ID NO: 2 of at least 80%, and RNA3 and RNA4 each have a sequence homology to SEQ ID NO: 1 of at least 80%.

Most preferably, RNA1 and RNA2 each have the sequence SEQ ID NO: 1, and RNA3 and RNA4 each have the sequence SEQ ID NO: 2.

Alternatively, RNA1 and RNA2 may each have the sequence SEQ ID NO: 2, and RNA3 and RNA4 may each have the sequence SEQ ID NO: 1.

A further aspect of the present invention concerns the oligonucleotide conjugate K or pharmaceutical composition according to the present invention for use in a method of treating or precluding a neoplasia, an infection and/or an uncontrolled immune response.

With regard to this aspect, the definitions and details above are correspondingly applicable.

In other words, the present invention also relates to a method of treating or precluding a neoplasia, an infection and/or an uncontrolled immune response in a patient, wherein the method comprises the administration of a suitable dose of the oligonucleotide conjugate K or a pharmaceutical composition to the patient.

As used herein, a patient in the broadest sense is understood to mean any individual to be treated with an oligonucleotide conjugate K of the invention or to be subjected to a preventative measure based on an oligonucleotide conjugate K of the invention. Therefore, the patient preferably has a neoplasia, an infection and/or an uncontrolled immune response or a risk of developing at least one of the above. The patient may be any animal, including a human. The patient is preferably a mammal (for example a human, a mouse, a rat, a cow, a pig, a dog, a cat or a horse, etc.). Most preferably, the patient is a human.

In the event of a neoplasia, an infection and/or an uncontrolled immune response, clinical symptoms may optionally be apparent, but do not necessarily occur, and may also occur in a latent (and therefore undiscovered) manner. A neoplasia, an infection and/or an uncontrolled immune response may occur in acute and/or chronic form (i.e. last, for example, for at least one month, at least six months or at least one year).

As used herein, a neoplasia in the broadest sense is understood to mean any tissue that leads to uncontrolled cellular growth. In many cases, a neoplasia tends to the formation of a tumor mass which may optionally be innervated by blood vessels. It is also possible, but not obligatory, for one or more metastases to occur.

A neoplasia in the context of the present invention may be any neoplasia as defined by the International Statistical Classification of Diseases and Related Health Problems 10^{th} Revision (ICD-10) as one of the ICD-10 classes C00-D48.

By way of example, a neoplasia in this connection may be a neoplasia selected from a malignant neoplasia (tumor) (ICD-10 classes C00-C97), an in situ neoplasia (ICD-10 classes D00-D09), a benign neoplasia (ICD-10 classes D10-D36) or a neoplasia of uncertain or unknown behavior (ICD-10 classes D37-D48).

In a preferred embodiment, the neoplasia is the genesis or presence of a tumor.

Preferably, the neoplasia is a cancer. Cancer may comprise one or more solid tumors and/or be a hematological disorder. Preferably, the neoplasia (in principle) responds to immunotherapeutic approaches, for example therapeutic antibodies directed against the neoplasia or analogs thereof.

Neoplasias can be divided and classified into different types. For instance, they can be characterized and classified according to the localization of their main body (more particularly, for instance, of a solid tumor) or else according to the original tissue origin of the neoplasia (i.e. to the tissue from which the neoplasia derives).

The neoplasia is preferably a malignant neoplasia (ICD-10 classes C00-C97). A neoplasia may, for instance, be selected from the group of malignant neoplasms at specific sites (ICD-10 classes C00-C75) (for example at the lip, oral cavity or pharynx (ICD-10 classes C00-C14), in a digestive organ (ICD-10 classes C15-C26), in a respiratory organ or other intrathoracic organs (ICD-10 classes C30-C39), in bones or articular cartilage (ICD-10 classes C40-C41), in skin (ICD-10 classes C43-C44), in mesothelial tissue or soft tissue (ICD-10 classes C45-C49), in the breast (ICD-10-Klasse C50), in female genital organs (ICD-10 classes C51-C58), in male genital organs (ICD-10 classes C60-C63), in a urinary organ (ICD-10 classes C64-C68), in the eye, brain or another part of the central nervous system (ICD-10 classes C69-C72) or in the thyroid or other endocrine glands (ICD-10 classes C73-C75)), malignant neoplasms of ill-defined, secondary and unspecific sites (ICD-10 classes C76-C80), malignant neoplasms of the lymphatic, hemopoietic and related tissue (ICD-10 classes C81-C96) and malignant neoplasms as primary tumors at multiple sites (ICD-10 class C97).

The neoplasia may be selected from the group consisting of a carcinoma (i.e. cancer types that derive from epithelial cells, for example an adenocarcinoma, a squamous cell carcinoma, an anaplastic carcinoma, a large-cell or small-cell (pulmonary) carcinoma), a sarcoma (i.e. cancer types that derive from connective tissue, for example an Askin tumor, botryoid sarcoma, a chondrosarcoma, a Ewing sarcoma, a malignant hemangioendothelioma, malignant schwannoma, an osteosarcoma, a soft tissue sarcoma), a lymphoma and leukemia (i.e. cancer types that derive from hematopoietic (blood-forming) cells, for instance B-cell neoplasias, T-cell neoplasias, natural killer cell neoplasias, Hodgkin’s lymphoma, non-Hodgkin’s lymphoma, immunodeficiency-associated lymphoproliferative disorders, lymphocytic leukemia, myelogenous leukemia), a germ cell tumor (i.e. cancer types that derive from pluripotent cells from the sexual organs, for example a germinoma (including dysgerminoma and seminoma), a blastoma (i.e. cancer types that derive from immature precursor cells or embryonic tissue, for instance a hepatoblastoma, a medulloblastoma, a nephroblastoma, a neuroblastoma, a pancreatoblastoma, a pleuropulmonary blastoma, a retinoblastoma, a glioblastoma), a melanoma and precursors thereof (i.e. cancer types that derive from melanocytes, for example lentigo maligna, superficial spreading melanoma, acral lentiginous melanoma, mucosal melanoma, nodular melanoma, polypoid melanoma, desmoplastic melanoma, amelanotic melanoma, soft tissue melanoma), non-melanoma skin cancer (i.e. non-melanoma cancer types that derive from the skin, for example basal cell carcinoma, squamous cell carcinoma, dermatofibrosarcoma protuberans, Merkel cell carcinoma, Kaposi sarcoma, keratoacanthoma, spindle cell tumor) and a glioma (i.e. cancer types that derive from brain or spinal cord cells, for example ependymoma, astrocytoma, oligodendroglioma, brainstem glioma, optic nerve glioma, mixed glioma).

An infection in the context of the invention may be any infection. Infection should be understood in the broadest sense as a pathological condition which is caused by the invasion of a pathogen or multiple pathogens. The pathogen here is generally a biological pathogen. This may, for instance, be an infection by viruses, bacteria, fungi, viroids, prions, eukaryotic microorganisms, nematodes, roundworms, flatworms, tapeworms and/or arthropods (e.g. insects, mites, ticks, lice, fleas).

In a preferred embodiment, the infection is a viral infection.

A viral infection may be acute or chronic. Nonlimiting examples of viral pathogens are, for example, poxvirus, Ebola virus, Lassa virus, Marburg virus, adenovirus, Herpes simplex, type 1, Herpes simplex, type 2, Varicella zoster virus, Epstein-Barr virus, human cytomegalovirus, human herpesvirus, type 8, human papillomavirus, BK virus, JC virus, smallpox virus, hepatitis B virus, hepatitis C virus, hepatitis D virus, hepatitis E virus, human bocavirus, parvovirus B19, human astrovirus, Norwalk virus, coxsackievirus, hepatitis A virus, poliovirus, rhinovirus, severe acute respiratory syndrome virus (SARS), yellow fever virus, Dengue virus, West Nile virus, rubella virus, HIV-1, HIV-2, influenza virus, Guanarito virus, Junin virus, Machupo virus, Sabiá virus, Crimean-Congo hemorrhagic fever virus, measles virus, mumps virus, parainfluenza virus, rotavirus, orbivirus, oltivirus and Banna virus.

An uncontrolled immune response may be an overreaction or an underreaction of the immune system.

It will be apparent to the person skilled in the art that the dose which is administered to the patient is dependent on the nature of the ailment to be treated, the condition of the patient and the weight of the patient, the age of the patient and the nature and severity of the disorder.

The oligonucleotide conjugate K of the invention is preferably administered to a patient in the form of a pharmaceutical composition. Therapeutic efficacy and toxicity can be determined by standard protocols.

The pharmaceutical composition can be administered systemically, for example intraperitoneally, intramuscularly or intravenously, or locally, for example intranasally, subcutaneously, intradermally or intrathecally. In a preferred embodiment, the pharmaceutical composition is administered intradermally. For this purpose, it is possible to use a tattooing method as described in WO 2014/049079 (cf., for example, pages 13-15 thereof).

The oligonucleotide conjugate K may be stabilized in that it is part of a pharmaceutical composition.

Therefore, the present invention also relates to a pharmaceutical composition comprising an oligonucleotide conjugate K according to the present invention and a pharmaceutically acceptable vehicle.

A pharmaceutically acceptable vehicle may be any substance which, in the amount used, has no toxic effect on the patient and is therefore tolerable to the patient. For example, a pharmaceutically acceptable vehicle may be a solvent, for example water, dimethyl sulfoxide (DMSO), ethanol, vegetable oil, paraffin oil or a combination of two or more of these.

A pharmaceutical composition of the invention may optionally also comprise one or more detergent(s) (e.g. sodium laurylsulfate (SLS)/sodium dodecylsulfate (SDS)), one or more dye(s) (e.g. TiO₂, food dye), one or more vitamin(s), one or more salt(s) (e.g. sodium, potassium, magnesium, calcium and/or zinc salts), one or more humectants (e.g. sorbitol, glycerol, mannitol, propylene glycol, polydextrose), one or more enzyme(s), one or more preservatives (e.g. benzoic acid, methylparaben), one or more thickeners (e.g. carboxymethylcellulose (CMC), polyethylene glycol (PEG), sorbitol), one or more solubilizers, one or more emulsifiers, one or more fillers, one or more glossing agents, one or more separating agents, one or more antioxidants, one or more plant extracts, one or more stabilizers, one or more polymers (e.g. hydroxypropylmethacrylamide (HPMA), polyethyleneimines (PEI), carboxymethyl cellulose (CMC), polyethylene glycol (PEG)), one or more absorption mediators (e.g. polyethyleneimine (PEI), dimethyl sulfoxide (DMSO), a cell-penetrating peptide (CPP), a protein transduction domain (PTD), an antimicrobial peptide etc.), one or more antibodies, one or more flavorings (e.g. sweeteners), one or more fluorescent substances and/or one or more fragrances. It is clear that some substances may also fulfill multiple functions.

The oligonucleotide conjugate K of the invention may optionally also be embedded into a macromolecular structure (e.g. a liposome, a polymerosome, a micelle structure, an (unstructured) hardened polymer (e.g. Eudragit) etc.). This is generally accomplished in a noncovalent manner. Alternatively or additionally, the oligonucleotide conjugate K of the invention may also be bound covalently or noncovalently to a polypeptide, a polymer, a resin bead etc. Formulations of this kind can considerably prolong the half-life in vivo and the shelf life. In addition, it is possible in this way to considerably improve the absorption rate into the body and into cells.

Both in vivo and in vitro, the absorption rate into the body and into cells can be improved by formulation with and/or covalent attachment to absorption mediators (e.g. polyethyleneimine (PEI), dimethyl sulfoxide (DMSO), a cell-penetrating peptide (CPP), a protein transduction domain (PTD), an antimicrobial peptide etc.). Absorption into the cytosol of the target compounds is achieved as far as possible. Absorption mediators should affect the activity of the oligonucleotide conjugate K of the invention (for example the RIG-I activation) only insignificantly, if at all, or, in the form of a precursor compound (prodrug), enable the release of the active RNA ligand, for instance within the cytosol. Noncovalent or covalent absorption mediators may likewise be:
liposomes,
micelles,
polymeric cationic lipids such as polyethyleneimines,
monosaccharides, disaccharides and other carbohydrates, for example glucose, mannose, galactose, dextrins or cyclodextrins etc.,
nonionic surface-active substances such as polyethylene or polyethylene glycol,
hydrophilic polymers such as polyacrylates.

In addition, the formulation of the RNA ligand may comprise auxiliaries for stabilization of the RNA ligands, such as chelating agents, salts or RNase-inhibiting substances.

An oligonucleotide conjugate K of the invention may in principle also be used in vitro.

Both in the medical context and in the non-medical context, in vivo and in vitro, the oligonucleotide conjugate K of the present invention may be used as the sole active pharmaceutical ingredient or in combination with one or more other active pharmaceutical ingredient(s). These may, for instance, be active anti proliferative/antineoplastic and antiviral ingredients. For example, active ingredients of this kind may be selected from the list consisting of purine antagonists and pyrimidine base antagonists, polyclonal or monoclonal antibodies (e.g. rituximab, trastuzumab, cetuximab, bevacizumab, basiliximab, daclizumab), antimetabolites (e.g. 5-fluorouracil, azathioprine, 6-mercaptopurine, mercaptopurine, pyrimidines, thioguanine, fludarabine, floxuridine, cytosine arabinoside (cytarabine), pemetrexed, raltitrexed, pralatrexat, methotrexat), antiestrogens, hormones and hormone antagonists, alkylators (e.g. mechlorethamine, cyclophosphamide, chlorambucil, ifosfamide), platins (e.g. cisplatin, carboplatin, oxaliplatin), plant alkaloids and terpenoids (e.g. vinca alkaloids (vincristin, vinblastin, vinorelbin, vindesin), taxanes (e.g. paclitaxel), cytoxan), topoisomerase inhibitors (e.g. camptothecine: irinotecan, topotecan, etoposide, etoposide phosphate, teniposide), melphalan, antineoplastics (e.g. doxorubicin (adriamycin), doxorubicin lipo, epirubicin, bleomycin)), actinomycin D, aminoglutethimide, amsacrin, anastrozole, anthracycline, aromatase inhibitors, asparaginase, bexarotene, buserelin, busulfan, camptothecin derivatives, capecitabine, carmustine, cladribine, cytarabine, cytosine arabinoside, alkylating cytostatics, dacarbazine, daunorubicin, docetaxel, epirubicin, estramustine, etoposide, exemestan, fludarabine, fluorouracil, folic acid antagonists, formestan, gemcitabine, glucocorticoids, goserelin, hycamtin, hydroxyurea, idarubicin, irinotecan, letrozole, leuprorelin, lomustine, mercaptopurine, miltefosine, mitomycins, mitose inhibitors, mitoxantrone, nimustine, procarbazine, tamoxifen, temozolomide, teniposide, testolactone, thiotepa, topoisomerase inhibitors, treosulfan, tretinoin, triptorelin, trofosfamide, cytostatically active antibiotics, everolimus, pimecrolimus, tacrolimus, azithromycin, spiramycin, sirolimus (rapamycin), roxithromycin, ascomycin, bafilomycin, erythromycin, midecamycin, josamycin, concancamycin, clarithromycin, troleandomycin, folimycin, tobramycin, mutamycin, dactinomycin, dactinomycin, rebeccamycin, A statin (e.g. cerivastatin, simvastatin, lovastatin, somatostatin, fluvastatin, nystatin, rosuvastatin, atorvastatin, pravastatin, pitavastatin, pentostatin), 4-hydroxyoxycyclophosphamide, bendamustine, thymosin α-1, aclarubicin, fludarabine 5′ -di hydrogen phosphate, hydroxycarbamide, aldesleukin, pegaspargase, cepharanthine, epothilone A and B, azathioprine, mycophenolate, mofetil, c-myc antisense, b-myc antisense, camptothecin, melanocyte stimulating hormone (α-MSH), activated protein C, IL-1 β inhibitor, fumaric acid and esters thereof, dermicidin, calcipotriol, taclacitol, lapachol, β-lapachone, podophyllotoxin, betulin, podophyllic acid, 2-ethylhydrazide, sagramostim, (rhuGM-CSF), peginterferon α-2b, lenograstim (r-HuG-CSF), filgrastim, macrogol, cephalomannin, selectin (cytokine antagonist), CETP inhibitor, cadherins, cytokinine inhibitors, COX inhibitor (e.g. COX-2 or COX-3 inhibitor), angiopeptin, ciprofloxacin, fluroblastin, BFGF antagonists, probucol, prostaglandins, 1,11-dimethoxyxanthin-6-one, 1-hydroxy-11-methoxyxanthin-6-one, scopoletin, colchicine, NO donors, pentaerythrityl tetranitrate, sydnone imines, S-nitroso derivatives, staurosporin, β-estradiol, α-estradiol, estriol, estrone, ethinylestradiol, fosfestrol, medroxyprogesterone, estradiol cypionates, estradiol benzoates, tranilast, kamebakaurin, verapamil, ciclosporin A, paclitaxel and derivatives thereof, for instance 6-α-hydroxypaclitaxel, baccatin, taxoter, mofebutazone, acemetacin, diclofenac, lonazolac, dapsone, O-carbamoylphenoxyacetate, lidocaine, ketoprofen, mefenamic acid, piroxicam, meloxicam, chloroquine phosphate, penicillamine, hydroxychloroquine, auranofin, sodium aurothiomalate, oxaceprole, celecoxib, β-sitosterol, ademetionine, myrtecaine, polidocanol, nonivamide, levomenthol, benzocaine, escin, elipticin, calbiochem D-24851, colcemid, cytochalasin A-E, indanocine, nocodazole, bacitracin, vitronectin receptor antagonists, azelastin, free nucleic acids, nucleic acids incorporated into virus transmitters, other DNA or RNA fragments, plasminogen activator inhibitor-1, plasminogen activating inhibitor-2, antisense oligonucleotide, VEGF inhibitors, IGF-1, active ingredients from the group of the antibiotics, for instance cefadroxil, cefazolin, cefaclor, cefoxitin, gentamicin, penicillins, dicloxacillin, oxacillin, sulfonamide, metronidazole, antithrombotics, argatroban, aspirin, abciximab, synthetic antithrombin, bivalirudin, Coumadin, enoxaparin, Gpllb/Illa platelet membrane receptor, antibodies against factor Xa inhibitor, heparin, hirudin, R-hirudin, PPACK, protamine, prourokinase, streptokinase, warfarin, urokinase, vasodilators, dipyramidole, trapidil, nitroprussides, PDGF antagonists, triazolopyrimidine, seramine, ACE inhibitors, captopril, cilazapril, lisinopril, enalapril, losartan, thioprotease inhibitors, prostacyclin, vapiprost, interferon-α, -β and -γ, histamine antagonists, serotonin blockers, apoptosis inhibitors, apoptosis regulators, NF-kB or Bcl-xL antisense oligonucleotides, halofuginone, nifedipine, tocopherol, molsidomine, tea polyphenols, epicatechin gallate, epigallocatechin gallate, boswellic acid and derivatives thereof, leflunomide, anakinra, etanercept, sulfasalazine, tetracycline, triamcinolone, procainimide, retinoic acid, quinidine, disopyramide, flecainide, propafenone, sotalol, amiodarone, natural and synthetic steroids, for instance bryophyllin A, inotodiol, maquiroside A, mansonine, strebloside, hydrocortisone, betamethasone, dexamethasone, fenoprofen, ibuprofen, indomethacin, naproxen, phenylbutazone, acyclovir, ganciclovir, zidovudine, antimycotics, clotrimazole, flucytosine, griseofulvin, ketoconazole, miconazole, terbinafin, chloroquine, mefloquine, quinine, natural terpenoids, hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, apocymarin, aristolochic acid, anopterine, hydroxyanopterine, anemonine, protoanemonine, berberine, cheliburin chloride, cicutoxin, sinococulin, combrestatin A and B, cudraisoflavone A, curcumin, dihydronitidine, nitidine chloride, 12-beta-hydroxypregnadiene-3,20-dione, bilobol, ginkgol, ginkgolic acid, helenalin, indicine, indicine N-oxide, lasiocarpine, inotodiol, glycoside 1a, justicidin A and B, larreatin, malloterin, mallotochromanol, isobutyrylmallotochromanol, marchantin A, maytansine, lycoridicine, margetine, pancratistatin, liriodenine, bisparthenolidine, oxoushinsunine, aristolactam All, periplocoside A, ghalakinoside, deoxypsorospermin, psychorubin, ricin A, sanguinarine, manwu wheat acid, methylsorbifolin, chromones from Spathelia, stizophyllin, akagerine, dihydrousambarensine, hydroxyusambarine, strychnopentamine, strychnophyllin, usambarine, usambarensine, daphnoretin, lariciresinol, methoxylariciresinol, syringaresinol, umbelliferone, afromoson, acetylvismione B, deacetylvismione A, vismione A and B), active antiangiogenic ingredients (e.g. carboxyamidotriazole, TNP-470, CM101, suramin, SU5416, thrombospondin, VEGFR antagonists, angiostatic steroids + heparin), matrix metalloprotease inhibitors, 2-methoxyestradiol, tecogalan, tetrathiomolybdate, thalidomide, thrombospondin, angiopoietin 2, angiostatin (e.g. TSP-1 and TSP-2 angiostatin), endostatin, vasostatin, canstatin, calreticulin, platelet factor-4, TIMP and CDAI, Meth-1 and Meth-2, kinase inhibitors (e.g. imatinib, imatinib mesylat, gefitinib, erlotinib, pazopanib, apatinib), proteasome inhibitors (e.g. bortezomib), PARP inhibitors (e.g. iniparib, olaparib), and combinations of two or more thereof.

It is also possible to employ radiation therapy and hence also to use iodine-131, lutetium-177, strontium-89, samarium (¹⁵³Sm), lexidronam and/or yttrium-90.

Therefore, a further aspect of the present invention relates to the use of an oligonucleotide conjugate K according to the present invention for induction of proinflammatory cytokines and/or for inducement of apoptosis in vitro.

With regard to this aspect, the above definitions and details are correspondingly applicable.

In this case, the oligonucleotide conjugate K of the invention can be added, for example, to cells in a cell culture. Having been propagated after a suitable incubation time, these can then secrete proinflammatory cytokines and/or become apoptotic.

A preferred example of a proinflammatory cytokine is interferon alpha (INFα), as also measured experimentally (see examples section).

The oligonucleotide conjugate K of the invention can be obtained in any desired manner. Compared to other oligonucleotide conjugates having individual similar structural features, however, particular synthetic advantages also arise.

Therefore, yet a further aspect of the present invention relates to a method of producing an oligonucleotide conjugate K according to the present invention, comprising the following steps:
(ia) parallel solid-phase synthesis of protected precursors of RNA1 and RNA2 on a common polymeric support, wherein the protected precursors of RNA1 and RNA2 are each bonded to the support by an equivalent terminus;
(iia) deprotecting the termini of the protected precursors from step (ia) that are remote from the polymeric support;
(iiia) linking an activated precursor of the bivalent linker B to the free termini of the terminally deprotected precursors of RNA1 and RNA2 from step (iia) or activated precursor from the terminally deprotected precursors of RNA1 and RNA2 from step (iia) to a bivalent linker B to give a conjugate RNA1-B-RNA2;
(iva) detaching and deprotecting the conjugates RNA1-B-RNA2 from step (iiia);
(va) purifying the conjugates RNA1-B-RNA2 from step (iva); and
(via) adding suitable proportions of RNA3 and RNA4 to give the conjugate from step (va) under conditions that allow the complementary strands to fit together.

With regard to this aspect, the above definitions and details are correspondingly applicable.

The person skilled in the art is able to conduct steps (ia) and (iia) by means of standard methods of production of oligonucleotides by means of solid-phase synthesis. The linking in step (iiia) depends on the bivalent linker B chosen in the particular case. In this case too, the person skilled in the art will resort to routine methods. Usually, in this case, either the bivalent linker B and/or the free termini of the terminally deprotected precursors of RNA1 and RNA2 are activated chemically by means of a coupling agent and then coupled. Detachment, deprotection and purification in steps (iva) and (va) are effected by routine methods from the solid-phase synthesis of oligonucleotides. The addition of suitable proportions of RNA3 and RNA4 (step (via)) is preferably effected in a substantially neutral aqueous environment (pH 6-7.5) at temperatures between 4° C. and 75° C., such that the complementary strands can fit together efficiently.

Yet a further aspect of the present invention relates to an alternative method of producing an oligonucleotide conjugate K according to the present invention, comprising the following steps:
(ib) providing a bivalent linker B bound to a solid phase;
(iib) parallel solid-phase synthesis of protected precursors of RNA1 and RNA2 onto the bivalent linker B bonded to a solid phase from step (ib), wherein the protected precursors of RNA1 and RNA2 are each bonded covalently to the bivalent linker B by an equivalent terminus;
(iiib) cleaving and deprotecting the conjugates obtained from step (iib) RNA1-B-RNA2;
(ivb) purifying the conjugates RNA1-B-RNA2 from step (iiib); and
(vb) adding suitable proportions of RNA3 and RNA4 to the conjugate from step (ivb) under conditions that allow the complementary strands to fit together.

With regard to this aspect, the above definitions and details are correspondingly applicable.

The bivalent linker B bonded to a solid phase (step (ib)) can be provided in any desired manner. It will be apparent to the person skilled in the art that the exact chemical procedure depends on the bivalent linker B used in the particular case. The person skilled in the art is aware of numerous standard methods for coupling. Steps (iib)-(ivb) can be effected by means of standard procedures in solid-phase synthesis of oligonucleotides. The addition of suitable proportions of RNA3 and RNA4 (step (via)) is preferably effected in a substantially neutral aqueous environment (pH 6-7.5) at temperatures between 4° C. and 75° C., such that the complementary strands can fit together efficiently. Preference is given to heating briefly in order to separate single strands from one another, and then lowering the temperature in order to fit the complementary strands together.

A further alternative and preferred embodiment relates to a method of producing an oligonucleotide conjugate K as shown in FIGS. 5 and 6. A method of this kind comprises the following steps:
(ic) parallel solid-phase synthesis of protected precursors of RNA1 and RNA2 on a common polymeric support, wherein the protected precursors of RNA1 and RNA2 are each bonded to the support by an equivalent terminus, wherein the precursors of RNA1 and RNA2 may optionally bear one or more additional linker structures L1 and/or L2 at the termini remote from the polymeric support;
(iic) partial deprotection of (about) 50% of the termini of the protected precursors from step (ic) that are remote from the polymeric support;
(iiic) monovalently linking an activated precursor of a bivalent linker structure B* to a free terminus of a terminally deprotected precursor from step (iic) or monovalently linking an activated precursor of a terminally deprotected precursor from step (iic) to a bivalent linker structure B* to give solid-phase-bound conjugates RNA1-B* and/or RNA2-B*;
(ivc) deprotecting the terminal end of the hitherto non-deprotected precursors;
(vc) conjugating the second, hitherto unbound binding site of a bivalent linker structure B* from the solid-phase-bound conjugates RNA1-B* and/or RNA2-B* to the deprotected terminal end of a precursor deprotected after step (ivc) to give a conjugate RNA1-B-RNA2;
(vic) detaching and deprotecting the conjugates RNA1-B-RNA2 from step (vc);
(viic) purifying the conjugates RNA1-B-RNA2 from step (vib); and
(viiic) adding suitable proportions of RNA3 and RNA4 to the conjugate from step (viib) under conditions that allow the complementary strands to fit together.

Ultimately, the bivalent linker structure B*, optionally together with L1 and/or L2, forms the bivalent linker B.

Preferably, in this case, RNA1 and RNA2 are identical, such that the parallel deprotection can also be effected in such a way that conditions are chosen under which, statistically, 50% of the solid-phase-bound precursors from step (ic) can also be used without special protecting group strategies. In that case, RNA1 and RNA2 preferably bear the same protecting group.

Alternatively, it is possible to work with orthogonal protecting group strategies (for example with Fmoc, Boc, (MeO)₂-trityl protecting groups etc.). In this case, it is then also possible to selectively link RNA1 via the bivalent linker B to RNA2. RNA1 and RNA2 in that case bear different protecting groups selectively detachable under different conditions and hence selectively deprotect RNA1 or RNA2 in each case. In this case, RNA1 and RNA2 are preferably in an equimolar ratio.

The deprotected functional groups that can be linked to one another can also be referred to as donor and acceptor.

By way of example, the linkage of the terminal ends of the precursors of RNA1 and RNA2 that are remote from the solid phase can be linked to one another (steps (iiic)-(vc)) via an H-phosphonate method as shown in FIGS. 5 and 6. If such a method is used, this method optionally also allows further derivatizations that can be inferred from the reactivity of the P(III) linker. The corresponding numerous options (for example reactions with sulfur compounds, oxidative amidation etc.) are known to those skilled in the art.

The bivalent linker B used here may preferably be phosphate or a phosphite derivative.

Optional additional linker structures L1 and/or L2 at the termini of RNA1 or RNA2 that are remote from the polymeric support may in principle be any desired bivalent structures. Preferably, structures of this kind comprise not more than 20 carbon atoms. By way of example, additional linker structures L1 and/or L2 of this kind may be ethylene glycols (e.g. hexaethylene glycol or triethylene glycol), amino acids, C₁₋₁₀-alkyl spacers or the like.

The efficiency of linkage reactions of this kind between two co-reactants fixed to a common polymeric support (solid-phase) is typically comparatively high. Moreover, the solid-phase synthesis of the precursors of RNA1 and RNA2 typically proceeds in good yields and high purity. The yield and purity of the conjugate RNA1-B-RNA2 and ultimately also the yield and purity of the oligonucleotide conjugate K of the present invention in such a synthesis strategy may likewise be particularly high.

As already set out above, the person skilled in the art may resort to common art knowledge in respect of the practical execution of the individual steps.

The examples and figures shown hereinafter are intended to serve to illustrate the invention, but not to restrict the scope of protection of the claims.

### BRIEF DESCRIPTION OF THE FIGURES

FIG. 1 shows a reaction scheme for the synthesis of RNA-5′-X4-5′-RNA (4, X4 = triethylene glycol linker). a) 50 mM 2-chloro-4H-1,3,2-benzodioxaphosphorin-4-one in dioxane/pyridine (3:1 v/v), 30 min; b) 1 M triethylammonium bicarbonate, 30 min; c) 0.1 M pivaloyl chloride, 2 mM triethylene glycol in acetonitrile/pyridine (1:1 v/v), 5 h d) 0.1 M iodine solution in THF/pyridine/water, 10 min.

FIG. 2 shows RP-LC/MS analyses of a RNA-5′-X4-5′-RNA synthesis (sequence: GACGCUGACCCUGAAGUUCAUCUU, X4 = triethylene glycol linker, GFP2_s2n55_T3EG) before (a) and after (b) purification. What is shown is the UV profile of the analysis of an RNA-5′-X4-5′-RNA synthesis when separated on an Acquity UPLC OST column (Waters, C18, 1.7 µm, 2.1 x 55 mm). Gradient: 7% B for 1.5 min, 7-15% B in 4.75 min, 15-40% B in 4.5 min, buffer A: 16.6 mM triethylamine, 100 mM HFIP, 10% methanol, buffer B: 16.6 mM triethylamine, 100 mM HFIP, 95% methanol. ESI-based mass determination of the relevant signals: a) 5′-OH-RNA (RT 4.10 min, 3.65%, calcd MW: 7590 Da, found MW: 7587 Da); 5′-H-p-RNA (RT 4.24 min, 1.60%, calcd MW: 7653 Da, found MW: 7652 Da); 5′-OH-X4-RNA (RT 4.59 min, 0.33%, calcd MW: 7802 Da, found MW: 7800 Da); n.d. (RT 4.81 min, 3.8%, found MW: 7787 Da); RNA-5′-5′-RNA (RT 5.54 min, 2.66%, calcd MW: Da, found MW: 15238 Da); RNA-5′-X4-5′-RNA (RT 5.62 min, 13.6%, calcd MW: 15454 Da, found MW: 15450 Da); 5′-Piv-X4-RNA (RT 7.12 min, 35.4%, calcd MW: 7886 Da, found MW: 7883 Da). b) RNA-5′-5′-RNA (RT 5.61, 14.8%, calcd MW: Da, found MW: 15238 Da); RNA-5′-X4-5′-RNA (RT 5.70 min, 63.3%, calcd MW: 15454 Da, found MW: 15450 Da).

FIG. 3 shows a schematic diagram of the synthesis strategies for discontinuous 5′-5′-, 3′-3′- and 3′-5′-linked RNA dimer structures.

FIG. 4 shows, by way of example, the formation of 5′-5′-linked RNA dimers obtained from the 5′-triphosphate RNA synthesis.

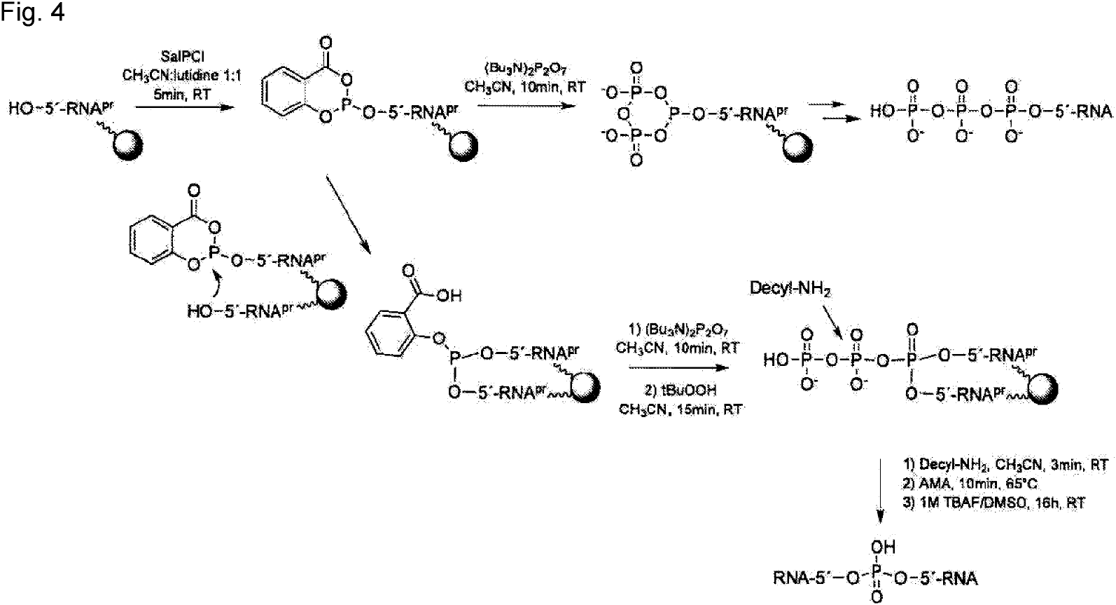

FIG. 5 shows a schematic diagram of an illustrative process for preparation of symmetrically 5′-5′- and 3′-3′-linked dimeric oligonucleotide conjugates K, where B represents the bivalent linker which, as shown by way of example here, is a phosphate linker. At first, the precursors of RNA1 and RNA2 are in solid-phase-bound form, such that the terminal remote from the solid-phase is protected (1). Then about half of the precursors are terminally deprotected (here: RNA1), while the other half remains protected (here: RNA2) (2). In a further step, the bivalent linker B is monovalently bound to the previously deprotected terminus (here OH₂PO₂) (3). There follows the deprotection of the other solid-phase-bound precursor strand (here: RNA2) (4). Thereafter, the two precursor strands are linked (here: RNA1 and RNA2 via the bivalent linker B) to give the conjugate RNA1-B-RNA2 (here: RNA1-O-PO-O-RNA2) (5). What follows here, by way of example, is oxidation to give a phosphoric diester, i.e. RNA1-O-P(O)(OH)-O-RNA2 (6), followed by the detachment of the conjugate (7) and the aggregation with the complementary oligonucleotide strands RNA3 and RNA4, which, as shown here, may be triphosphorylated (ppp) at a terminus (here: at the 5′ terminus) (8), which affords an oligonucleotide conjugate K of the present invention.

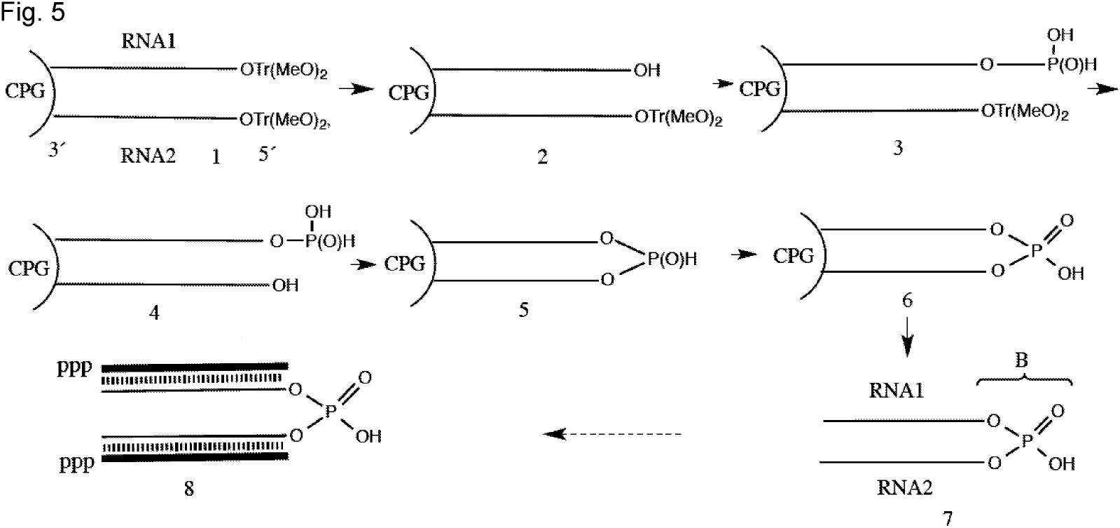

FIG. 6 shows a schematic diagram of an illustrative method of producing symmetrically 5′-5′- and 3′-3′-linked dimeric oligonucleotide conjugates K, where B represents the bivalent linker, which, as shown here by way of example, is a phosphate linker bonded via additional, non-nucleotide linker structures L1 and L2. The method illustrated in this figure proceeds like the method from FIG. 5, except that additional linker structures L1 and L2 between RNA1 and RNA2 and the phosphate linker are present, and these likewise become part of the bivalent linker B that ultimately consists of L1, phosphate and L2.

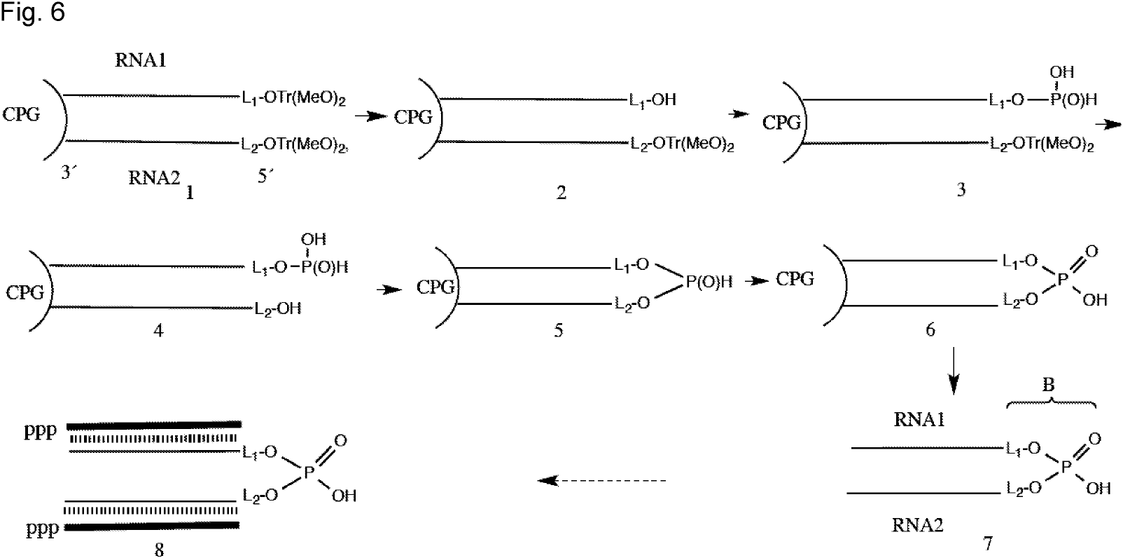

### EXAMPLES

### Material and Methods

### Reagents

The reagents used were purchased from Sigma-Aldrich or Roth and used without further purification.

### RIG-I Stimulation Assay in Human Peripheral Blood Mononuclear Cells (PBMCs)

For stimulation tests with human PBMCs, the cells were isolated and plated out directly before the start of the experiment. The cells were pretreated with chloroquine in order to avoid stimulation of the endosomal TLR receptors and to enable selective analysis of the RIG-I activity. Stimulation with GFP2_as (single-strand RNA) as TLR ligand here controlled suppression of the endosomal receptors. For the analysis of the double-stranded oligonucleotides, the complementary single strands were first hybridized by heating to 72° C. for two minutes and gradually cooling to room temperature. Subsequently, the stimuli were introduced into cells using Lipofectamin 2000 (Invitrogen) in the specified concentration series (the molar concentrations are each based on the amount of the 24mer units). After 20 h, the IFNα secretion was determined by means of ELISA. The stimulation assays were each conducted in a double determination for 2-4 donors.

### Oligonucleotides

5′—OH—Oligoribonucleotide syntheses were purchased from either of Biomers.net GmbH and Axolabs GmbH. The syntheses of 5′-3′-linked oligonucleotides were effected using DMT-2′-O-TBDMS-rC(ac), DMT-2′-O-TBDMS-rA(bz), DMT-2′-O-TBDMS-rG(ib) and DMT-2′-O-TBDMS-rU-phosphoramidites on the 0.2-1 µmolar scale with a CPG loading of 39-44 µmol/g.

Tetraethylene glycol and propyl linkers were introduced using the corresponding phosphoramidites. For the linear synthesis of 5′-5′- or 3′-3′-linked structures, sub-sequences were synthesized with the aid of the reverse 3′-DMT-rN-5′-CED-phosphoramidites (ChemGenes Corporation).

The parallel synthesis of 5′-5′- or 3′-3′-linked structures was effected on branching supports (ChemGenes Cooperation), which had a glycerol unit having two DMT-protected hydroxyl groups for the chain extension. For 5′-triphosphorylated sequences, the triphosphorylation and purification were effected as described above (WO2012/130886, Goldeck et al., 2014, Angew. Chem. 126:4782-4786).

TABLE 1

Sequences of some oligonucleotides used and structural units of the oligonucleotide conjugates K

| No. | Based on SEQ ID NO | Name | Sequence |
|---|---|---|---|
| 1 | 1 | GFP2_s | 5′-GACGCUGACCCUGAAGUUCAUCUU-3′ |
| 2 | 2 | GFP2_as | 5′-AAGAUGAACUUCAGGGUCAGCGUC-3′ |
| 3 | 1^{p} | ppp-GFP2_s | ppp-5′-GACGCUGACCCUGAAGUUCAUCUU-3′ |
| 4 | 2^{p} | ppp-GFP2_as | ppp-5′-AAGAUGAACUUCAGGGUCAGCGUC-3′ |
| 5 | 1^{#} | GFP2_s2n55 | 3′-UUCUACUUGAAGUCCCAGUCGCAG-5′-5′ -GACGCUGACCCUGAAGUUCAUCUU |
| 6 | 3 | GFP2_s2n53 | 5′-GACGCUGACCCUGAAGUUCAUCUU GACGCUGACCCUGAAGUUCAUCUU-3′ |
| 7 | 2⁺ | GFP2_as2n33 | 5′-AAGAUGAACUUCAGGGUCAGCGUC-3′-3′-CUGCGACUGGGACUUCAAGUAGAA-5′ |
| 8 | 1^{M1p} | ppp-GFP2_sOMe1 | ppp-5-gACGCUGACCCUGAAGUUCAUCUU-3′ |
| 9 | 1^{M1} | GFP2_sOMe1 | 5′-gACGCUGACCCUGAAGUUCAUCUU-3′ |
| 10 | 2^{M1} | GFP2_asOMe1 | 5′-aAGAUGAACUUCAGGGUCAGCGUC-3′ |
| 11 | 1^{M2} | GFP2_sOMe2 | 5′-GaCGCUGACCCUGAAGUUCAUCUU-3′ |
| 12 | 1^{M3} | GFP2_sOMe3 | 5′-GAcGCUGACCCUGAAGUUCAUCUU-3′ |
| 13 | 1^{M4} | GFP2_sOMe4 | 5′-GACqCUGACCCUGAAGUUCAUCUU-3′ |
| 14 | 1^{M5} | GFP2_sOMe5 | 5′-GACGcUGACCCUGAAGUUCAUCUU-3′ |
| 15 | 1^{M6} | GFP2_sOMe6 | 5′-GACGCuGACCCUGAAGUUCAUCUU-3′ |
| 16 | 1^{M7} | GFP2_sOMe7 | 5′-GACGCUgACCCUGAAGUUCAUCUU-3′ |
| 17 | 1^{M8} | GFP2_sOMe8 | 5′-GACGCUGaCCCUGAAGUUCAUCUU-3′ |
| 18 | 1^{M9} | GFP2_sOMe9 | 5′-GACGCUGAcCCUGAAGUUCAUCUU-3′ |
| 19 | 1^{M10} | GFP2_sOMe10 | 5′-GACGCUGACcCUGAAGUUCAUCUU-3′ |
| 20 | 2^{FAM} | GFP2_as5′-FAM | 5′-FAM-AAGAUGAACUUCAGGGUCAGCGUC-3′ |
| 21 | 2^{#} | GFP2_as2n55 | 3′-CUGCGACUGGGACUUCAAGUAGAA-5′- |
| | | | 5′-AAGAUGAACUUCAGGGUCAGCGUC-3′ |
| 22 | 2^{#L} | GFP2_as2n55_C3br | 3′-CUGCGACUGGGACUUCAAGUAGAA-5′-X1-5′-AAGAUGAACUUCAGGGUCAGCGUC-3′ |
| 23 | 2^{#L} | GFP2_as2n55_T4EG | 3′-CUGCGACUGGGACUUCAAGUAGAA-5′-X2-5′-AAGAUGAACUUCAGGGUCAGCGUC-3′ |
| 24 | 2^{+L} | GFP2_as2n33_C3br | 5′-AAGAUGAACUUCAGGGUCAGCGUC-3′-X1-3′-CUGCGACUGGGACUUCAAGUAGAA-5′ |
| 25 | 2~^{L} | GFP2_as2n53_C3 | 5′-AAGAUGAACUUCAGGGUCAGCGUC-3′-X3-5′-AAGAUGAACUUCAGGGUCAGCGUC-3′ |
| 26 | 1^{#L} | GFP2_s2n55_T3EG | 3′-UUCUACUUGAAGUCCCAGUCGCAG-5′-X4-5′-GACGCUGACCCUGAAGUUCAUCUU |

p with triphosphorylated 5′ terminus
# two copies of this sequence linked to one another via a 5′-5′ linkage
+ two copies of this sequence linked to one another via a 3′-3′ linkage
~ two copies of this sequence 3′-5′-linked to one another via a propyl linker
M1 where the nucleotide has a 2′—O—methylation at position 1
M2 where the nucleotide has a 2′—O—methylation at position 2
M3 where the nucleotide has a 2′—O—methylation at position 3
M4 where the nucleotide has a 2′—O—methylation at position 4
M5 where the nucleotide has a 2′—O—methylation at position 5
M6 where the nucleotide has a 2′—O—methylation at position 6
M7 where the nucleotide has a 2′—O—methylation at position 7
M8 where the nucleotide has a 2′—O—methylation at position 8
M9 where the nucleotide has a 2′—O—methylation at position 9
M10 where the nucleotide has a 2′—O—methylation at position 10
FAM 5′-terminally modified with FAM (= 6-carboxyfluorescein)
L linked via a bivalent linker B:
X1 = glycol linker,
X2 = tetraethylene glycol linker,
X3 = propyl linker,
X4 = triethylene glycol linker

### Results

### The Linkage of Oligoribonucleotide Duplexes via 5′-5′- or 3′-3′-phosphodiester bonds leads to highly active RIG-I ligands

It is known that 5′-triphosphate-dsRNAs serve as potent activators of the RIG-I immune sensor. In a comparative study, it was now assessed whether, as well as the triphosphate group, structural modifications also enable modulation of the RIG-I ligand properties of 5′-OH-RNAs. In a first example, the linkage of two 5′—OH—dsRNA 24mer units to give dimers via 5′-5′-, 3′-3′- and 3′-5′-phosphodiester bonds was the subject of a comparative study.

The model sequence chosen for the studies which follow was the 24mer ‘GFP2’ sequence. 48mer structures were obtained via standard RNA syntheses wherein, for the introduction of the 5′-5′ and 3′-3′ linkages, a sub-sequence was constructed in each case using the reverse RNA amidites (ChemGenes Corporation). Subsequently, the sequences were supplemented with the complementary 24mer opposing strands and checked for RIG-I activity in human PBMCs (tables 2 and 3). For the 5′-3′-linked dimer, as expected for 5′—OH—dsRNAs, very weak RIG-I activity was observed. By comparison, surprisingly, the linkage of the two 24mer units via 5′-5′- or 3′-3′-phosphodiester bonds led to highly active RIG-I ligands. Thus, these results introduce the 5′-5′ or 3′-3′ linkage of dsRNA units as a new RIG-I activity-enhancing structural element.

TABLE 2

Comparison of the RIG-I activity of 5′-5′- and 5′-3′-linked 5′—OH—dsRNA dimers

| RNA (nM) | IFNα (ng/ mL) | | | | | | | | | |
|---|---|---|---|---|---|---|---|---|---|---|
| | GFP2_as (single strand, Seq. No. 2) | | Medium | | ppp-GFP2 (Seq. No. 3/2) | | GFP2_s2n55 (Seq. No. 5/2) | | GFP2_s2n35 (Seq. No. 6/2) | |
| | MW | SD | MW | SD | MW | SD | MW | SD | MW | SD |
| 50.00 | 0.017 | 0.00 | 0.017 | 0.00 | 2.19 | 0.553 | 8.681 | 1.342 | 0.752 | 0.06 |
| 5.00 | | | | | 1.761 | 0.107 | 6.366 | 0.295 | 0.718 | 0.036 |
| 0.50 | | | | | 0.078 | 0.012 | 2.444 | 0.269 | 0.13 | 0.012 |

The compounds were introduced into chloroquine-treated human PBMCs in a titration series (50, 5, 0,5 nM) and the stimulatory activity was detected via measurement of the IFNα secretion after 20 h. The molar concentrations are based on the content of 24mer units. What are shown are the mean and standard deviation from the double determination for a representative donor (n = 4).

TABLE 3

Comparison of the RIG-I activity of 5′-5′- and 3′-3′-linked 5′—OH—dsRNA dimers

| RNA (nM) | IFNα (ng/ mL) | | | | | | | | | |
|---|---|---|---|---|---|---|---|---|---|---|
| | GFP2_as (single strand, Seq. No. 2) | | Medium | | ppp-GFP2 (Seq. No. 3/2) | | GFP2_s2n55 (Seq. No. 5/2) | | GFP2_as2n33 (Seq. No. 7/1) | |
| | MW | SD | MW | SD | MW | SD | MW | SD | MW | SD |
| 17.00 | 0.00 | 0.00 | 0.02 | 0.03 | 0.64 | 0.01 | 1.13 | 0.10 | 0.68 | 0.00 |
| 5.00 | | | | | 0.34 | 0.07 | 1.23 | 0.11 | 0.42 | 0.12 |
| 0.50 | | | | | 0.05 | 0.00 | 0.56 | 0.00 | 0.06 | 0.03 |

The RNA duplexes were introduced into chloroquine-treated human PBMCs in a titration series (17, 5, 0.5 nM) and the stimulatory activity was detected via measurement of the IFNα secretion after 20 h. The molar concentrations are based on the content of 24mer units. What are shown are the mean and standard deviation from the double determination for a representative donor (n = 4).

### The Immune Recognition of 5′-5′- and 3′-3′-linked dsRNAs by RIG-I

The novel symmetric 5′-5′- and 3′-3′-linked dimer ligands was examined in detail hereinafter with reference to structure-activity analyses, in order to ensure and give a more detailed characterization of recognition by the RIG-I receptor. For the recognition of short RNA duplexes by RIG-I, it is common knowledge that, at first, the 5′-end is recognized by the C-terminal domain. Then the helicase domain is bound to the RNA duplex, and there is a change in conformation and activation through release of the CARD domains which mediate the further signal transmission. It was possible in each case to clarify the binding details of the RIG-I-CTD to 5′-triphosphate-dsRNA and also to 5′—OH—dsRNA from crystal structures (Wang et al. 2010, Nature Structural & Molecular Biology, Nat Struct Mol Biol. 2010 Jul;17(7):781-787; Lu et al. 2010, Nucleic Acids Res. 2011 Mar;39(4):1565-1575). In both cases, a stacking interaction of the 5′-terminal base pair with the phenylalanine residue F853 and a hydrogen bond between the 2′-hydroxyl group of the first nucleotide and the histidine residue H830 constitute essential binding contacts. For the recognition both of 5′-ppp-dsRNA and of 5′—OH—dsRNA, as well as an intact base pair at the 5′ end, a free 2′—OH group at the terminal 5′ nucleotide is thus essential. Therefore, these known binding contacts were subjected to a comparative study for substitution studies with ppp-dsRNA and the novel 5′-5′- and 3′-3′-linked 5′—OH—dsRNA dimers (table 4).

It was possible to confirm that a 2′—OMe substitution on the first nucleotide at the 5′ triphosphate terminus of ppp-dsRNA ligands (pppGFP2_sOMe1) prevents RIG-I CTD attachment by steric hindrance of the interaction with H830 and suppresses the interferon response in hPBMCs. At the opposite, inner 5′—OH end of ppp-dsRNA, a 2′—OMe substitution at the terminal nucleotide (pppGFP2_asOMe1) has only a minor influence on the stimulatory activity. This confirms that, in the RIG-I recognition of ppp-dsRNA, the opposite end of the duplex from the triphosphate group is not bound by the CTD domain. The analysis of the 2′—OMe substitution effects was thus suitable for interrogating ultimate duplex recognition by RIG-I, and both the effect of the substitution of the outwardly directed ends (GFP2_s2n55_asOMe1) and methylation on the 5′-terminal nucleotides of the ‘inner’ phosphate-bonded ends (GFP2_as2n33_sOMe1) was examined in the 5′-5′- and 3′-3′-linked dimer. Complete absence of the interferon response on substitution of the outer ends and substantial tolerance at the inner ends gave a clear suggestion that the immune response of the dimer structures is mediated exclusively by RIG-I and the RIG-I activation is initiated by the RIG-I CTD attachment at the outer 5′—OH ends.

In the context of further sedition experiments on the 5′-terminal nucleotides, the influence of steric hindrance was examined. Substitution by 5′-fluorescein as a bulky radical at the outer ends (GFP2_s2n55_asFAM) led to a complete loss of activity (table 4). On the other hand, to increase the RIG-I affinity of the outer ends, a 5′-triphosphate group was introduced (GFP2_s2n55s_as-ppp). This further potentiated the activity of the GFP2 dimer, and even in low concentrations maximum amounts of IFNα were measured (table 5). These observations thus further support the hypothesis of the necessity of a RIG-I CTD attachment to the outer ends of the dimer for the stimulatory activity.

After these studies relating to the first binding contact between the RIG-I CTD domain and the 5′ terminus of the ligands, in the next step, the further RIG-I activation with binding of the helicase domains was examined by substitution analysis on the first 10 nucleotides. In a comparative analysis, 2′—O—methylation in the 5′—OH—dsRNA 24mer and in the 5′-5′-linked 48mer ligands was analyzed, with substitution at the outer ends of the duplex in the case of the dimer (tables 6 and 7). In accordance with the observations so far, the 24mers generally showed much lower activities than the corresponding dimer compounds. Both compounds correspondingly showed a relative decrease in the interferon response in the case of 2′—O methylations at nucleotides 4-9. These results support a common recognition mechanism of the 5′—OH—dsRNA 24mer compounds and the 5′-5′-linked dimer compounds by RIG-I with comparable binding contacts to the RIG-I CTD domain and also to the duplex-recognizing helicase domain.

TABLE 4

Substitution effects of 2′—O—methylation at the terminal nucleotides

| RNA (nM) | IFNα (ng/ mL) | | | | | | | | | | | | | | | | | |
|---|---|---|---|---|---|---|---|---|---|---|---|---|---|---|---|---|---|---|
| | GFP2_as (single strand, Seq. No. 2) | | Medium | | pppGFP2 (Seq. No. 3/2) | | pppGFP2 _asOMe1 (Seq. No. 3/10) | | pppGFP2 _sOMe1 (Seq. No. 8/2) | | GFP2 _s2n55 (Seq. No. 5/2) | | GFP2_s2n 55 _asOMet (Seq. No. 5/10) | | GFP2 _as2n33 (Seq. No. ⅐) | | GFP2 _as2n33_s OMe1 (Seq. No. 9/7) | |
| | MW | SD | MW | SD | MW | SD | MW | SD | MW | SD | MW | SD | MW | SD | MW | SD | MW | SD |
| 17.00 | 0.00 | 0.00 | 0.00 | 0.00 | 3.36 | 0.15 | 1.43 | 0.12 | 0.06 | 0.03 | 3.25 | 0.29 | 0.00 | 0.00 | 3.14 | 0.18 | 2.46 | 0.20 |
| 5.00 | | | | | 1.49 | 0.22 | 0.45 | 0.17 | 0.03 | 0.04 | 2.82 | 0.21 | 0.00 | 0.00 | 2.38 | 0.10 | 1.52 | 0.00 |
| 1.70 | | | | | 0.01 | 0.01 | 0.00 | 0.00 | 0.01 | 0.01 | 0.39 | 0.55 | 0.00 | 0.00 | 0.01 | 0.01 | 0.03 | 0.04 |

2′—OMe substitutions were introduced at the 5′-terminal nucleotides of the 5′-triphosphate-dsRNA ligand pppGFP2 and the 5′-5′- and 3′-3′-linked duplexes GFP2_s2n55 and GFP2_as2n33, and the stimulatory activity was introduced into chloroquine-treated PBMCs in a titration series. IFNα production was determined by means of ELISA after 20 h. The molar concentrations are based on the content of 24mer units. What are shown in each case are mean values and standard deviation from the double determination for a representative donor (n = 4).

TABLE 5a

Effect of steric hindrance on the outer 5′-terminal nucleotides of 5′-5′-linked dimer ligands on the RIG-I activity

| RNA (nM) | IFNα (ng/ mL) | | | | | | | |
|---|---|---|---|---|---|---|---|---|
| | GFP2_as (single strand, Seq. No. 2) | | Medium | | ppp-GFP2 (Seq. No. 3/2) | | GFP2_s2n55_asFAM (Seq. No. 5/20) | |
| | MW | SD | MW | SD | MW | SD | MW | SD |
| 50.00 | 0.07 | 0.02 | 0.10 | 0.03 | 20.62 | 2.57 | 0.12 | 0.02 |
| 5.00 | | | | | 9.57 | 0.27 | 0.09 | 0.04 |
| 0.50 | | | | | 0.42 | 0.17 | 0.04 | 0.00 |

The hybridization of the 5′-5′-linked dimer GFP2_s2n55 with a 5′-fluorescein-substituted complementary opposing strand gave a ligand variant with steric hindrance at the outer dimer ends. RIG-I activity was detected in chloroquine-treated PBMCs by determination of the IFNα production. The molar concentrations are based on the content of 24mer units. What are shown in each case are mean values and standard deviation from the double determination for a representative donor (n = 4).

TABLE 5b

Effect of 5′-triphosphorylation at the outer 5′-terminal nucleotides of 5′-5′-linked dimer ligands on RIG-I activity

| RNA (nM) | IFNα (ng/ mL) | | | | | | | | | |
|---|---|---|---|---|---|---|---|---|---|---|
| | GFP2_as (single strand, Seq. No. 2) | | Medium | | ppp-GFP2 (Seq. No. 3/2) | | GFP2_s2n55 (Seq. No. 5/2) | | GFP2_s2n55_as-ppp (Seq. No. 5/4) | |
| | MW | SD | MW | SD | MW | SD | MW | SD | MW | SD |
| 50.00 | 0.11 | 0.01 | 0.05 | 0.01 | 28.16 | 3.11 | 24.44 | 8.93 | 36.52 | 2.29 |
| 5.00 | | | | | 3.76 | 0.02 | 13.06 | 1.75 | 35.83 | 1.54 |
| 0.50 | | | | | 0.07 | 0.00 | 0.13 | 0.01 | 29.85 | 1.43 |

The 5′-5′-linked dimer GFP2_s2n55 was hybridized with a 5′-triphosphorylated complementary opposing strand and introduced into chloroquine-treated PBMCs. After 20 h, IFNα production was detected. The molar concentrations are based on the content of 24mer units. What are shown in each case are mean values and standard deviation from the double determination for a representative donor (n = 4).

TABLE 6

2′—O—Methyl substitution effects at nucleotides 1-10 in RIG-I recognition of 5′—OH—dsRNA ligands

| RNA (nM) | IFNα (ng/ mL) | | | | | | | | | | | | | |
|---|---|---|---|---|---|---|---|---|---|---|---|---|---|---|
| | GFP2_as (single strand, Seq. No. 2) | | Medium | | GFP2 (Seq. No. ½) | | GFP2_sOMe1 (Seq. No. 9/2) | | GFP2_sOMe2 (Seq. No. 11/2) | | GFP2_sOMe3 (Seq. No. 12/2) | | GFP2_sOMe4 (Seq. No. 13/2) | |
| | MW | SD | MW | SD | MW | SD | MW | SD | MW | SD | MW | SD | MW | SD |
| 50.00 | 0.00 | 0.00 | 0.00 | 0.00 | 6.61 | 1.15 | 0.25 | 0.09 | 0.61 | 0.18 | 4.75 | 0.35 | 0.97 | 0.25 |
| 5.00 | | | | | 0.08 | 0.02 | 0.00 | 0.00 | 0.00 | 0.00 | 0.08 | 0.02 | 0.00 | 0.00 |

IFNα (ng/ mL)

| GFP2_sOMe5 (Seq. No. 14/2) | | GFP2_sOMe6 (Seq. No. 15/2) | | GFP2_sOMe7 (Seq. No. 16/2) | | GFP2_sOMe8 (Seq. No. 17/2) | | GFP2_sOMe9 (Seq. No. 18/2) | | GFP2_sOMe10 (Seq. No. 19/2) | |
|---|---|---|---|---|---|---|---|---|---|---|---|
| MW | SD | MW | SD | MW | SD | MW | SD | MW | SD | MW | SD |
| 1.04 | 0.05 | 0.47 | 0.02 | 0.22 | 0.04 | 0.83 | 0.10 | 1.08 | 0.15 | 4.75 | 0.24 |
| 0.00 | 0.00 | 0.00 | 0.00 | 0.02 | 0.02 | 0.00 | 0.00 | 0.00 | 0.00 | 0.02 | 0.02 |

5′-OH-GFP2 compounds with 2′—O methylations at nucleotides 1-10 were hybridized with the complementary 24mer opposing strand and used for stimulation of chloroquine-treated human PBMCs. Transfection was effected with RNA concentrations of 50 and 5 nM (based on the amount of 24mer units) and, after 20 h, the amounts of IFNα in the supernatant were quantified by ELISA. What are shown are the mean and standard deviation from the double determination for a representative donor (n = 2).

TABLE 7

2′—O—Methyl substitution effects at nucleotides 1-10 in RIG-I recognition of 3′-3′-linked 5′—OH—dsRNA dimer ligands

| RNA (nM) | IFNα (ng/ mL) | | | | | | | | | | | | | | | | | | | |
|---|---|---|---|---|---|---|---|---|---|---|---|---|---|---|---|---|---|---|---|---|
| | GFP2_as (single strand, Seq. No. 2) | | | | Medium | | GFP2_as2n 55 (Seq. No. 1/21 | | | GFP2_as2n 55_sOMe1 (Seq. No. 9/21) | | | GFP2_as2n 55_sOMe2 (Seq. No. 11/21) | | GFP2_as2n 55_sOMe3 (Seq. No. 12/21) | | | | GFP2_as2n 55_sOMe4 (Seq. No. 13/21) | |
| | MW | | SD | | MW | SD | MW | SD | | MW | | SD | MW | SD | MW | | SD | | MW | SD |
| 50.00 | 0.00 | | 0.00 | | 0.00 | 0.00 | 15.33 | 9.45 | | 0.00 | | 0.00 | 13.02 | 2.90 | 22.54 | | 8.59 | | 17.26 | 3.19 |
| 5.00 | | | | | | | 11.32 | 1.87 | | 0.00 | | 0.00 | 6.13 | 0.87 | 24.17 | | 0.39 | | 8.95 | 0.42 |
| IFNα (ng/ mL) | | | | | | | | | | | | | | | | | | | | |
| GFP2_as2n5 5_sOMe5 (Seq. No. 14/21) | | | | GFP2_as2n5 5_sOMe6 (Seq. No. 15/21) | | | GFP2_as2 n55_sOMe 7 (Seq. No. 16/21) | | | | GFP2_as2n5 5_sOMe8 (Seq. No. 17/21) | | | GFP2_as2n5 5_sOMe9 (Seq. No. 18/21) | | | | GFP2_as2n5 5_sOMe10 (Seq. No. 19/21) | | |
| MW | | SD | | MW | | SD | MW | | SD | | MW | | SD | MW | | SD | | MW | | SD |
| 11.88 | | 3.04 | | 13.44 | | 0.25 | 8.61 | | 1.01 | | 12.26 | | 1.62 | 14.51 | | 0.20 | | 19.06 | | 1.82 |
| 8.16 | | 0.37 | | 10.76 | | 0.30 | 4.13 | | 0.27 | | 7.94 | | 0.22 | 15.49 | | 1.67 | | 21.01 | | 2.80 |

5′-OH-GFP2 compounds with 2′—O methylations at nucleotides 1-10 were hybridized with the complementary 5′-5′-linked 48mer opposing strand and used for stimulation of chloroquine-treated human PBMCs. Transfection was effected with RNA concentrations of 50 and 5 nM (based on the amount of 24mer units) and, after 20 h, the amounts of IFNα in the supernatant were quantified by ELISA. What are shown are the mean and standard deviation from the double determination for a representative donor (n = 2).

### Variation of the RNA Sequence in RNA-5′-p-5′-RNA Recognition

In the first examples, dimer formation of the GFP2 model sequence at the otherwise triphosphorylated 5′ terminus (5′-GACG..., GFP2_s2n55 and GFP2_as2n33) was examined, in which the opposite ends (5′-AAGA...) in the dimer are directed outward and hence mediate recognition by RIG-I. As the first sequence variation, by virtue of the alternative linkage of the GFP2 duplexes via the 5′ ends of the antisense strand (GFP2_2n55as), the opposite 5′ termini (5′-GACG...) were directed outward. On analysis of the RIG-I activity in human PBMCs, it was possible to observe an equally strong interferon response analogously to the triphosphate compounds irrespective of the sequence for the dimer compounds (table 8). For this dimer too, it was possible by additional triphosphorylation of the outer ends (pppGFP2_as2n55) to further potentiate the RIG-I activity. These results suggest general applicability of the activity-promoting 5′-5′ and 3′-3′ structure motifs to further RNA dimer sequences.

TABLE 8

Comparison of the dimer compounds GFP2_2n55s and GFP2_2n55as.

| RNA (nM) | IFNα (ng/ mL) | | | | | | | | | | | | | |
|---|---|---|---|---|---|---|---|---|---|---|---|---|---|---|
| | GFP2_as (single strand, Seq. No. 2) | | ppp-GFP2 | | GFP2_ppp-as (Seq. No. 3/2) | | GFP2_s2n55 (Seq. No. 5/2) | | GFP2_s2n55_as-ppp (Seq. No. 5/4) | | GFP2_as2n55 (Seq. No.1/21) | | pppGFP2_as2n55 (Seq. No. 3/21) | |
| | MW | SD | MW | SD | MW | SD | MW | SD | MW | SD | MW | SD | MW | SD |
| 5.00 | 0.16 | 0.00 | 14.08 | 1.00 | 17.88 | 1.85 | 19.10 | 1.11 | 22.45 | 0.54 | 24.86 | 2.58 | 25.73 | 1.50 |
| 1.60 | | | 9.73 | 0.53 | 13.18 | 0.19 | 17.31 | 1.50 | 24.35 | 1.54 | 19.05 | 0.19 | 25.05 | 1.46 |
| 0.50 | | | 4.04 | 0.45 | 2.13 | 0.91 | 7.44 | 0.42 | 19.76 | 2.81 | 6.94 | 4.40 | 20.76 | 2.92 |
| 0.17 | | | 0.27 | 0.06 | 0.20 | 0.06 | 0.41 | 0.14 | 11.47 | 1.08 | 0.65 | 0.37 | 14.59 | 3.81 |
| 0.05 | | | 0.16 | 0.00 | 0.16 | 0.00 | 0.12 | 0.06 | 8.74 | 0.82 | 0.12 | 0.06 | 9.88 | 2.01 |
| 0.01 | | | 0.08 | 0.11 | 0.20 | 0.17 | 0.20 | 0.06 | 5.39 | 0.48 | 0.04 | 0.06 | 5.68 | 0.24 |

The dimer compounds GFP2_s2n55 and GFP2_as2n55 were hybridized with the complementary OH-RNA opposing strand and the pppRNA opposing strand and used in comparison with pppGFP2 and GFP2_as-ppp for stimulation of chloroquine-treated human PBMCs. The transfection was effected as a titration series (5, 1.6, 0,5, 0.16, 0.05, 0.005 nM, based on the concentration of 24 units) and, after 20 h, the amounts of IFNα in the supernatant were quantified by ELISA. What are shown are the mean and standard deviation from the double determination for a representative donor (n = 2).

### The Introduction of Linker Structures at the 5′-3′, 5′-5′ and 3′-3′ Bond Between the dimer units is tolerated by RIG-I

In the further study of ligand requirements, the effect of different linker structures on the 5′-5′ or 3′-3′ linkage of the dimers was analyzed, using the compounds GFP2_as2n55 and GFP2_as2n33 as example sequences. Firstly, the introduction of a C3 unit was examined. The synthesis approach chosen here was the parallel synthesis of the two 24mer units on a CPG-bound glycerol linker (branching 3′-Icaa-CPG, ChemGenes Corporation). The support material enables, proceeding from the linker unit, either 5′-5′ linkage using the reverse RNA amidites for the RNA synthesis or 3′-3′ linkage using the standard RNA amidites. As example compounds, the compounds GFP2_as2n55_C3br and GFP2_as2n33_C3br were thus synthesized. In addition, a tetraethylene glycol unit was inserted into the GFP2_as2n55 compound as an example of a longer linker structure (GFP2_as2n55_T4EG). The synthesis here was implemented as a continuous RNA synthesis using the appropriate tetraethylene glycol phosphoramidite, employing the reverse RNA amidites. As well as these 5′-5′ and 3′-3′ linkages, the effect of the introduction of linker structures was also examined in the context of a regular 5′-3′-linked dimer. As an example synthesis for this purpose, a propanediol linker was introduced during the continuous RNA synthesis (GFP2_as2n53_C3). On testing in human PBMCs, all the linker-containing dimer compounds synthesized showed high RIG-I activity, and a comparable to slightly weaker interferon response was achieved compared to the 5′-5′- and 3′-3′-phosphate-linked dimers (tables 9 and 10). It was possible to further increase the activity of the linker-linked dimers by an additional introduction of 5′-triphosphate groups. These results show that the introduction of linker structures into the 5′-5′- and 3′-3′-linked RNA dimers with retention of the high RIG-I activity is possible, and even the interruption of continuous 5′-3′-linked dimers by linker structures can entail an increase in activity. Thus, the RIG-I activity-promoting structural element can extend to the group of the linker-bridged 5′-3′, 5′-5′ and 3′-3′ linkages. This enables flexible and versatile synthesis of the corresponding ligands, with enablement not only of continuous 48mer synthesis but also of parallel synthesis of the 24mer units at branching CPG units as a further synthesis approach.

TABLE 9

The activation of RIG-I by 5′-5′-linked RNA-p linker-p-RNA compounds

| RNA (nM) | IFNα (ng/ mL) | | | | | | | | | | | | | | | | | |
|---|---|---|---|---|---|---|---|---|---|---|---|---|---|---|---|---|---|---|
| | GFP2_as (single strand, Seq. No. 2) | | Medium | | ppp-GFP2 (Seq. No. 3/2) | | GFP2_as2n 55 (Seq. No. 5/21) | | GFP2_as2n5 5_C3br (Seq. No. 1/22) | | GFP2_as2n55 _T4EG (Seq. No. 1/23) | | pppGFP2_as 2n55 (Seq. No. 3/21) | | pppGFP2_as 2n55_C3br (Seq. No. 3/22) | | pppGFP2_as 2n55_T4EG (Seq. No. 3/23) | |
| | MW | SD | MW | SD | MW | SD | MW | SD | MW | SD | MW | SD | MW | SD | MW | SD | MW | SD |
| 50.00 | 0.02 | 0.03 | 0.00 | 0.00 | 27.93 | 2.07 | 23.81 | 5.00 | 21.80 | 1.78 | 21.80 | 2.07 | 22.38 | 4.33 | 22.38 | 4.33 | 22.52 | 1.35 |
| 5.00 | | | | | 21.50 | 2.02 | 27.52 | 3.42 | 14.32 | 2.93 | 12.27 | 3.72 | 26.02 | 1.40 | 26.02 | 1.40 | 24.66 | 0.72 |
| 0.50 | | | | | 1.70 | 0.57 | 2.78 | 0.90 | 0.16 | 0.07 | 0.07 | 0.05 | 24.49 | 2.21 | 24.49 | 2.21 | 20.99 | 7.26 |
| 0.05 | | | | | 0.04 | 0.00 | 0.02 | 0.03 | 0.00 | 0.00 | 0.02 | 0.03 | 19.69 | 0.53 | 19.69 | 0.53 | 13.16 | 0.91 |

The GFP2_as2n55 dimers linked via a glycol linker (C3br), via a propane linker (C3) or via a tetraethylene linker (T4EG) were hybridized with the complementary 5′—OH and 5′-triphosphate opposing strands. Reference substances used were the pppGFP2 duplex and the phosphodiester-linked GFP2_s2n55 duplex. The compounds were used for stimulation of chloroquine-treated human PBMCs. Transfection was effected in a titration series (50, 5, 0.5, 0.005 nM) and, after 20 h, the amounts of IFNα in the supernatant were quantified by ELISA. The concentrations specified are based on the content of 24mer units. What are shown are the mean and standard deviation from the double determination for a representative donor (n=2).

TABLE 10

The activation of RIG-I by 3′-3- and 3′-5′-linked RNA-p linker-p-RNA compounds

| RNA (nM) | IFNα (ng/ mL) | | | | | | | | | | | |
|---|---|---|---|---|---|---|---|---|---|---|---|---|
| | GFP2_as (single strand, Seq. No. 2) | | Medium | | ppp-GFP2 (Seq. No. 3/2) | | GFP2_as2n3 3 (Seq. No. ⅐) | | GFP2_as2n3 3_C3br (Seq. No. 1/24) | | GFP2_as2n5 3_C3 (Seq. No. 1/25) | |
| | MW | SD | MW | SD | MW | SD | MW | SD | MW | SD | MW | SD |
| 17.00 | 0.00 | 0.00 | 0.00 | 0.00 | 16.15 | 0.95 | 19.66 | 1.15 | 15.06 | 2.22 | 17.92 | 0.08 |
| 5.00 | | | | | 12.94 | 1.52 | 12.98 | 3.76 | 11.00 | 2.96 | 14.66 | 4.85 |
| 1.70 | | | | | 9.86 | 1.10 | 5.00 | 0.95 | 5.00 | 0.95 | 4.35 | 0.50 |

For the 3′-3′ and 3′-5′ linkage of dimers, the effect of a glycol linker (C3br) and of a propane linker (C3) on RIG-I activity was examined. The dimers were hybridized with the complementary opposing strands and used for stimulation of chloroquine-treated PBMCs. After 20 h, the amounts of IFNα in the supernatant were quantified by ELISA. The concentrations reported are based on the content of 24mer units. What are shown are the mean and standard deviation from the double determination for a representative donor (n=2).

### The Synthesis of Dimeric RIG-I Ligands by Linker-mediated 5′-5′ Linkage Of support-bound RNA sequences

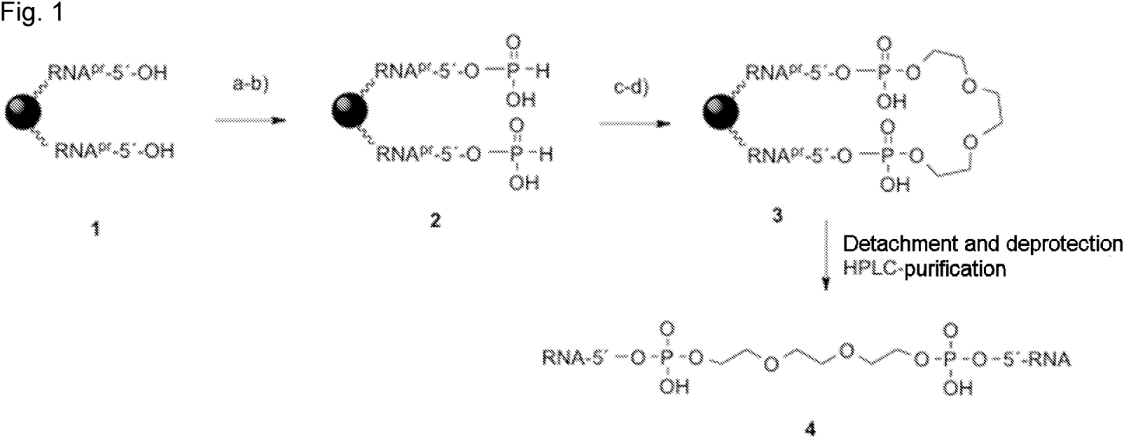

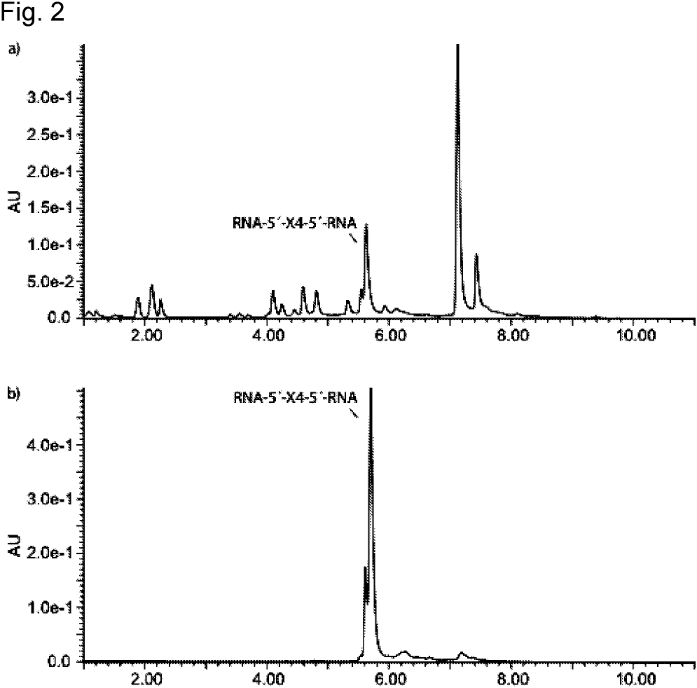

As well as the parallel synthesis of the sub-sequences on branching support materials, the possibility of the linkage of support-bound 5′-OH-RNA sequences constitutes an attractive synthesis strategy for dimeric RIG-I ligands. For this purpose, the phosphitylation and hydrolysis of support-bound 5′-OH-RNA sequences to give the H-phosphonate derivatives was followed by the reaction with bifunctional linkers in the presence of pivaloyl chloride and oxidation of the phosphite to phosphate groups. This synthesis approach was applied successfully, for example, to the 5′-5′ linkage of the GFP2 sequence via a triethylene glycol linker (GFP2_2n55_T3EG) (FIGS. 1 and 2).

The fully protected, support-bound 5′-OH-GFP2 model sequence (1 µmol) was first washed with an anhydrous pyridine/dioxane solution (1:3, v/v, 4 mL). Subsequently, a freshly made-up 50 mM 2-chloro-4H-1,3,2-benzodioxaphosphorin-4-one solution (2 mL, 100 µmol) in dry dioxane/pyridine (3:1, v/v) was passed through the column with the aid of plastic syringes. The solution was repeatedly contacted with the synthesis column by means of the plastic syringe by drawing it up and expelling it for a reaction time of 30 minutes. Subsequently, the synthesis column was washed with acetonitrile (3 mL). In the next step, the synthesis column was incubated with an aqueous 1 M triethylammonium bicarbonate solution (1 mL) for 30 min and hence the support-bound 5′—H—phosphonate-RNA was obtained. Thereafter, the synthesis column was washed with acetonitrile (3 mL) and acetonitrile/pyridine (1:1, v/v, 3 mL). For the linker-mediated linkage, a solution of pivaloyl chloride (12 µL, 0.1 mmol) and triethylene glycol (0.27 µL, 2 µmol) was made up in acetonitrile/pyridine (1:1, v/v, 1 mL) and passed through the synthesis column. An incubation time of 5 h was followed by a further wash step with acetonitrile (3 mL) and oxidation with a 0.1 M iodine solution in THF/pyridine/water (2 mL) for 10 min. Subsequently, the synthesis product was washed with acetonitrile (3 x 3 mL), dried in an argon stream and cleaved from the support material and deprotected under standard conditions. The 5′-5′-linked dimer product was purified via HPLC chromatography on an anion exchange column (Source15Q 4.6/100, GE Healthcare) in a sodium perchlorate gradient (20 mM Tris-HCl, pH8, 1 mM EDTA, 80-320 mM NaClO₄) and desalinated by means of a HiTrap column (GE Healthcare). The integrity of the product obtained was confirmed via RP-LC/MS analysis.

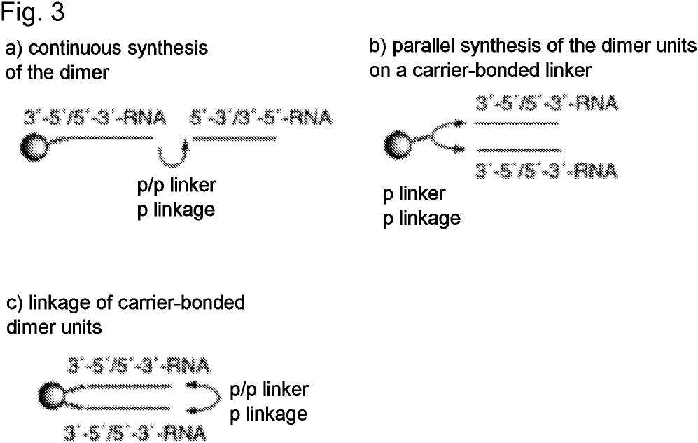

The RIG-I activity of the GFP2_s2n55_T3EG product obtained was ascertained in a stimulation experiment in human PBMCs. Slightly higher interferon responses compared to ppp-GFP2 and interferon responses comparable to GFP2_s2n55 were measured (FIG. 3). It was thus possible to confirm that linker-linked 5′-5′-dimer structures also constitute highly active RIG-I ligands.

In summary, it was thus possible to demonstrate, for the synthesis of RIG-I ligand structures bonded via 5′-5′- and 3′-3′-phosphodiester bonds and via 5′-5′, 3′-3′ or 3′-5′ linker structures, the continuous synthesis and parallel synthesis of the dimer units at branching units or in combination with a post-synthetic linkage (FIG. 3). Particularly the parallel synthesis of the short dimer units can enable more efficient and less costly synthesis methods and the provision of the ligands in high purity.

TABLE 11

The RIG-I activity of a triethylene glycol linker-linked 5′-5′ dimer

| RNA (nM) | IFNα (ng/ mL) | | | | | | | | | |
|---|---|---|---|---|---|---|---|---|---|---|
| | GFP2_as (single strand, Seq. No. 2) | | Medium | | ppp-GFP2 (Seq. No. 3/2) | | GFP2_s2n5 5 (Seq. No. 5/2) | | GFP2_s2n5 5_T3EG (Seq. No. 26/2) | |
| | MW | SD | MW | SD | MW | SD | MW | SD | MW | SD |
| 50.00 | 0.00 | 0.00 | 0.00 | 0.00 | 7.37 | 0.25 | 7.86 | 0.98 | 6.38 | 0.93 |
| 17.00 | | | | | 5.69 | 0.00 | 8.43 | 0.00 | 8.05 | 1.47 |
| 5.00 | | | | | 5.94 | 0.00 | 7.72 | 1.67 | 6.23 | 0.09 |
| 1.70 | | | | | 2.23 | 0.38 | 5.71 | 1.30 | 3.20 | 1.44 |

The GFP2_s2n55_T3EG dimer 5′-5′-linked via a triethylene glycol linker was hybridized with the complementary opposing strand and examined for RIG-I activity in chloroquine-treated PBMCs by comparison with the ppp-GFP2 duplex and with the GFP2_s2n55 dimer duplex directly 5′-5′-linked via a phosphodiester bond. The concentration figures are based on the amount of 24mer units. What are shown are the mean values and standard deviations of a double determination for a representative donor (n = 2).

1-20. (canceled)

21. A method of treating or precluding a tumor or a viral infection, or both, in a subject, wherein the method comprises administering an effective amount of an oligonucleotide conjugate K comprising the structure 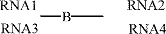
or a pharmaceutically compatible salt thereof to the subject, wherein:
a) RNA1 is a first ribonucleic acid or an analog or derivative thereof of at least six nucleotides in length;
b) RNA2 is a second ribonucleic acid or an analog or derivative thereof of at least six nucleotides in length;
c) RNA3 is a third ribonucleic acid or an analog or derivative thereof of at least six nucleotides in length, which forms at least five complementary base pairs with RNA1;
d) RNA4 is a fourth ribonucleic acid or an analog or derivative thereof of at least six nucleotides in length, which forms at least five complementary base pairs with RNA2; and
e) B is a bivalent phosphodiester linker having a molecular weight of not more than 1500 Da that covalently bonds the 5′ terminus of RNA1 to the 5′ terminus of RNA2 or the 3′ terminus of RNA1 to the 3′ terminus of RNA2,
wherein RNA3 and RNA4 are not covalently bonded to one another,
wherein the oligonucleotide conjugate K is an activator of the cytosolic helicase retinoic acid-inducible gene I (RIG-I), and
wherein RNA1 and RNA3, and RNA2 and RNA4, each have no overhang of the 5′-terminal nucleotide residues and an overhang of the 3′-terminal nucleotide residues of not more than five nucleotides.

22. The method of claim 21, wherein the sequence of RNA2 is identical to the sequence of RNA1.

23. The method of claim 21, wherein the sequence of RNA3 is identical to the sequence of RNA4.

24. The method of claim 21, wherein B is a bivalent linker that
a) covalently bonds the 5′ terminus of RNA1 to the 5′ terminus of RNA2, and wherein RNA3 and RNA4 each have, at their 5′ termini, triphosphate residues, triphosphate analog residues or free hydroxyl groups; or
b) covalently bonds the 3′ terminus of RNA1 to the 3′ terminus of RNA2, and wherein RNA1 and RNA2 each have, at their 5′ termini, triphosphate residues, triphosphate analog residues or free hydroxyl groups.

25. The method of claim 21, wherein RNA1, RNA2, RNA3 and RNA4 each have a length of between 10 and 50 nucleotides.

26. The method of claim 25, wherein RNA1, RNA2, RNA3 and RNA4 each have a length of between 15 and 40 nucleotides.

27. The method of claim 26, wherein RNA1, RNA2, RNA3 and RNA4 each have a length of between 19 and 30 nucleotides.

28. The method of claim 27, wherein RNA1, RNA2, RNA3 and RNA4 each have a length of between 20 and 25 nucleotides.

29. The method of claim 21, wherein RNA1 and RNA3, and RNA2 and RNA4, each have no overhang of the 5′-terminal nucleotide residues and an overhang of the 3′-terminal nucleotide residues of not more than four nucleotides.

30. The method of claim 21, wherein RNA1 and RNA3, and RNA2 and RNA4, each have no overhang of the 5′-terminal nucleotide residues and an overhang of the 3′-terminal nucleotide residues of not more than three nucleotides.

31. The method of claim 21, wherein RNA1 and RNA3, and RNA2 and RNA4, each have no overhang of the 5′-terminal nucleotide residues and an overhang of the 3′-terminal nucleotide residues of not more than two nucleotides.

32. The method of claim 21, wherein RNA1 and RNA3, and RNA2 and RNA4, each have no overhang of the 5′-terminal nucleotide residues and an overhang of the 3′-terminal nucleotide residues of not more than one nucleotide.

33. The method of claim 21, wherein RNA1 and RNA3, and RNA2 and RNA4, each have no overhang of the 5′-terminal nucleotide residues and no overhang of the 3′-terminal nucleotide residues.

34. The method of claim 21, wherein RNA1 is fully complementary to RNA3 and RNA2 is fully complementary to RNA4.

35. The method of claim 21, wherein the sequence of RNA2 is identical to the sequence of RNA1 and the sequence of RNA3 is identical to the sequence of RNA4, wherein each of the sequences of RNA1, RNA2, RNA3, and RNA4 are of equal length, and wherein RNA1 is fully complementary to RNA3 and RNA2 is fully complementary to RNA4.

36. The method of claim 21, wherein:
RNA1 or RNA3 have a sequence homology of at least 80% to SEQ ID NO: 1; or
each of RNA1 and RNA2 or RNA3 and RNA4 have a sequence homology of at least 80% to SEQ ID NO: 1 and each of RNA3 and RNA4 or RNA1 and RNA2 have a sequence homology of at least 80% to SEQ ID NO: 2.

37. The method of claim 36, wherein each of RNA1 and RNA2 or RNA3 and RNA4 have a sequence homology of at least 80% to SEQ ID NO: 1.

38. The method of claim 21, wherein:
RNA1 or RNA3 have a sequence homology of at least 80% to SEQ ID NO: 1and
each of RNA1 and RNA2 or RNA3 and RNA4 have a sequence homology of at least 80% to SEQ ID NO: 1 and each of RNA3 and RNA4 or RNA1 and RNA2 have a sequence homology of at least 80% to SEQ ID NO: 2.

39. The method of claim 38, wherein each of RNA1 and RNA2 or RNA3 and RNA4 have a sequence homology of at least 80% to SEQ ID NO: 1.

40. The method of claim 21, wherein the oligonucleotide conjugate K is comprised in a pharmaceutical composition further comprising a pharmaceutically acceptable vehicle.

41. The method of claim 21, wherein the oligonucleotide conjugate K is used in combination with one or more active antiproliferative or antiviral ingredients.

42. A method for increasing secretion of interferon alpha by a cell comprising contacting the cell with an oligonucleotide conjugate K comprising the structure 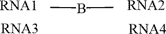
or a pharmaceutically compatible salt thereof, wherein:
a) RNA1 is a first ribonucleic acid or an analog or derivative thereof of at least six nucleotides in length;
b) RNA2 is a second ribonucleic acid or an analog or derivative thereof of at least six nucleotides in length;
c) RNA3 is a third ribonucleic acid or an analog or derivative thereof of at least six nucleotides in length, which forms at least five complementary base pairs with RNA1;
d) RNA4 is a fourth ribonucleic acid or an analog or derivative thereof of at least six nucleotides in length, which forms at least five complementary base pairs with RNA2; and
e) B is a bivalent phosphodiester linker having a molecular weight of not more than 1500 Da that covalently bonds the 5′ terminus of RNA1 to the 5′ terminus of RNA2 or the 3′ terminus of RNA1 to the 3′ terminus of RNA2,
wherein RNA3 and RNA4 are not covalently bonded to one another,
wherein the oligonucleotide conjugate K is an activator of the cytosolic helicase retinoic acid-inducible gene I (RIG-I), and
wherein RNA1 and RNA3, and RNA2 and RNA4, each have no overhang of the 5′-terminal nucleotide residues and an overhang of the 3′-terminal nucleotide residues of not more than five nucleotides.


SEQUENCE LISTING (26 sequences)

SEQ ID NO: 1 - RNA, 24 nt, synthetic construct
  misc_feature 1..24: note = GFP2 model sequence
  source 1..24: mol_type = other RNA
gacgctgaccctgaagttcatctt

SEQ ID NO: 2 - RNA, 24 nt, synthetic construct
  misc_feature 1..24: note = GFP2 model sequence antisense
  source 1..24: mol_type = other RNA
aagatgaacttcagggtcagcgtc

SEQ ID NO: 3 - RNA, 48 nt, synthetic construct
  misc_feature 1..48: note = GFP2 model sequence dimer
  misc_feature 1: note = triphosphorylated 5' terminus
  source 1..48: mol_type = other RNA
ttctacttgaagtcccagtcgcaggacgctgaccctgaagttcatctt

SEQ ID NO: 4 - RNA, 24 nt, synthetic construct
  misc_feature 1..24: note = Synthetic
  misc_feature 1: note = triphosphorylated 5' terminus
  source 1..24: mol_type = other RNA
gacgctgaccctgaagttcatctt

SEQ ID NO: 5 - RNA, 24 nt, synthetic construct
  misc_feature 1..24: note = Synthetic
  misc_feature 1: note = triphosphorylated 5' terminus
  source 1..24: mol_type = other RNA
aagatgaacttcagggtcagcgtc

SEQ ID NO: 6 - DNA, 48 bp, synthetic construct
  misc_feature 1..48: note = Synthetic
  misc_feature 1..24: note = 3'-5' orientation
  source 1..48: mol_type = other DNA
  modified_base 1: mod_base = OTHER; note = Uracil
  modified_base 2: mod_base = OTHER; note = Uracil
  modified_base 4: mod_base = OTHER; note = uracil
  modified_base 7: mod_base = OTHER; note = Uracil
  modified_base 8: mod_base = OTHER; note = Uracil
  modified_base 13: mod_base = OTHER; note = Uracil
  modified_base 19: mod_base = OTHER; note = Uracil
  modified_base 30: mod_base = OTHER; note = Uracil
  modified_base 36: mod_base = OTHER; note = Uracil
  modified_base 41: mod_base = OTHER; note = Uracil
  modified_base 42: mod_base = OTHER; note = Uracil
  modified_base 45: mod_base = OTHER; note = Uracil
  modified_base 47: mod_base = OTHER; note = Uracil
  modified_base 48: mod_base = OTHER; note = Uracil
ttctacttgaagtcccagtcgcaggacgctgaccctgaagttcatctt

SEQ ID NO: 7 - DNA, 48 bp, synthetic construct
  misc_feature 1..48: note = Synthetic
  misc_feature 25..48: note = 3'-5' orientation
  source 1..48: mol_type = other DNA
  modified_base 5: mod_base = OTHER; note = Uracil
  modified_base 10: mod_base = OTHER; note = Uracil
  modified_base 11: mod_base = OTHER; note = Uracil
  modified_base 23: mod_base = OTHER; note = Uracil
  modified_base 26: mod_base = OTHER; note = Uracil
  modified_base 32: mod_base = OTHER; note = Uracil
  modified_base 38: mod_base = OTHER; note = Uracil
  modified_base 39: mod_base = OTHER; note = Uracil
  modified_base 44: mod_base = OTHER; note = Uracil
aagatgaacttcagggtcagcgtcctgcgactgggacttcaagtagaa

SEQ ID NO: 8 - RNA, 24 nt, synthetic construct
  misc_feature 1..24: note = Synthetic
  misc_feature 1: note = triphosphorylated 5' terminus
  source 1..24: mol_type = other RNA
gacgctgaccctgaagttcatctt

SEQ ID NO: 9 - DNA, 24 bp, synthetic construct
  misc_feature 1..24: note = Synthetic
  misc_feature 1: note = 2'-O-methylation
  source 1..24: mol_type = other DNA
  modified_base 6: mod_base = OTHER; note = Uracil
  modified_base 12: mod_base = OTHER; note = Uracil
  modified_base 17: mod_base = OTHER; note = Uracil
  modified_base 18: mod_base = OTHER; note = Uracil
  modified_base 21: mod_base = OTHER; note = Uracil
  modified_base 23: mod_base = OTHER; note = Uracil
  modified_base 24: mod_base = OTHER; note = Uracil
gacgctgaccctgaagttcatctt

SEQ ID NO: 10 - RNA, 24 nt, synthetic construct
  misc_feature 1..24: note = Synthetic
  misc_feature 1: note = 2'-O-methylation
  source 1..24: mol_type = other RNA
aagatgaacttcagggtcagcgtc

SEQ ID NO: 11 - RNA, 24 nt, synthetic construct
  misc_feature 1..24: note = Synthetic
  misc_feature 2: note = 2'-O-methylation
  source 1..24: mol_type = other RNA
gacgctgaccctgaagttcatctt

SEQ ID NO: 12 - RNA, 24 nt, synthetic construct
  misc_feature 1..24: note = Synthetic
  misc_feature 3: note = 2'-O-methylation
  source 1..24: mol_type = other RNA
gacgctgaccctgaagttcatctt

SEQ ID NO: 13 - RNA, 24 nt, synthetic construct
  misc_feature 1..24: note = Synthetic
  misc_feature 4: note = 2'-O-methylation
  source 1..24: mol_type = other RNA
gacgctgaccctgaagttcatctt

SEQ ID NO: 14 - RNA, 24 nt, synthetic construct
  misc_feature 1..24: note = Synthetic
  misc_feature 5: note = 2'-O-methylation
  source 1..24: mol_type = other RNA
gacgctgaccctgaagttcatctt

SEQ ID NO: 15 - RNA, 24 nt, synthetic construct
  misc_feature 1..24: note = Synthetic
  misc_feature 6: note = 2'-O-methylation
  source 1..24: mol_type = other RNA
gacgctgaccctgaagttcatctt

SEQ ID NO: 16 - RNA, 24 nt, synthetic construct
  misc_feature 1..24: note = Synthetic
  misc_feature 7: note = 2'-O-methylation
  source 1..24: mol_type = other RNA
gacgctgaccctgaagttcatctt

SEQ ID NO: 17 - RNA, 24 nt, synthetic construct
  misc_feature 1..24: note = Synthetic
  misc_feature 8: note = 2'-O-methylation
  source 1..24: mol_type = other RNA
gacgctgaccctgaagttcatctt

SEQ ID NO: 18 - RNA, 24 nt, synthetic construct
  misc_feature 1..24: note = Synthetic
  misc_feature 9: note = 2'-O-methylation
  source 1..24: mol_type = other RNA
gacgctgaccctgaagttcatctt

SEQ ID NO: 19 - RNA, 24 nt, synthetic construct
  misc_feature 1..24: note = Synthetic
  source 1..24: mol_type = other RNA
gacgctgaccctgaagttcatctt

SEQ ID NO: 20 - RNA, 24 nt, synthetic construct
  misc_feature 1..24: note = Synthetic
  misc_feature 1: note = FAM (= 6-carboxyfluorescein)
  source 1..24: mol_type = other RNA
aagatgaacttcagggtcagcgtc

SEQ ID NO: 21 - DNA, 48 bp, synthetic construct
  misc_feature 1..48: note = Synthetic
  misc_feature 1..24: note = 3'-5' orientation
  source 1..48: mol_type = other DNA
  modified_base 2: mod_base = OTHER; note = Uracil
  modified_base 8: mod_base = OTHER; note = Uracil
  modified_base 14: mod_base = OTHER; note = Uracil
  modified_base 15: mod_base = OTHER; note = Uracil
  modified_base 20: mod_base = OTHER; note = Uracil
  modified_base 29: mod_base = OTHER; note = Uracil
  modified_base 34: mod_base = OTHER; note = Uracil
  modified_base 35: mod_base = OTHER; note = Uracil
  modified_base 41: mod_base = OTHER; note = Uracil
  modified_base 47: mod_base = OTHER; note = Uracil
ctgcgactgggacttcaagtagaaaagatgaacttcagggtcagcgtc

SEQ ID NO: 22 - DNA, 48 bp, synthetic construct; identical to SEQ ID NO: 21
  misc_feature 1..48: note = Synthetic
  misc_feature 1..24: note = 3'-5' orientation
  misc_feature 24: note = glycol linker
  source 1..48: mol_type = other DNA
  modified_base 2: mod_base = OTHER; note = Uracil
  modified_base 8: mod_base = OTHER; note = Uracil
  modified_base 14: mod_base = OTHER; note = Uracil
  modified_base 15: mod_base = OTHER; note = Uracil
  modified_base 20: mod_base = OTHER; note = Uracil
  modified_base 29: mod_base = OTHER; note = Uracil
  modified_base 34: mod_base = OTHER; note = Uracil
  modified_base 35: mod_base = OTHER; note = Uracil
  modified_base 41: mod_base = OTHER; note = Uracil
  modified_base 47: mod_base = OTHER; note = Uracil

SEQ ID NO: 23 - DNA, 48 bp, synthetic construct; identical to SEQ ID NO: 21
  misc_feature 1..48: note = Synthetic
  misc_feature 1..24: note = 3'-5' orientation
  misc_feature 24: note = tetraethylene glycol linker
  source 1..48: mol_type = other DNA
  modified_base 2: mod_base = OTHER; note = Uracil
  modified_base 14: mod_base = OTHER; note = Uracil
  modified_base 8: mod_base = OTHER; note = Uracil
  modified_base 15: mod_base = OTHER; note = Uracil
  modified_base 20: mod_base = OTHER; note = Uracil
  modified_base 29: mod_base = OTHER; note = Uracil
  modified_base 34: mod_base = OTHER; note = Uracil
  modified_base 35: mod_base = OTHER; note = Uracil
  modified_base 41: mod_base = OTHER; note = Uracil
  modified_base 47: mod_base = OTHER; note = Uracil

SEQ ID NO: 24 - DNA, 48 bp, synthetic construct; identical to SEQ ID NO: 7
  misc_feature 1..48: note = Synthetic
  misc_feature 24: note = glycol linker
  misc_feature 25..48: note = 3'-5' orientation
  source 1..48: mol_type = other DNA
  modified_base 5: mod_base = OTHER; note = Uracil
  modified_base 10: mod_base = OTHER; note = Uracil
  modified_base 11: mod_base = OTHER; note = Uracil
  modified_base 17: mod_base = OTHER; note = Uracil
  modified_base 23: mod_base = OTHER; note = Uracil
  modified_base 26: mod_base = OTHER; note = Uracil
  modified_base 32: mod_base = OTHER; note = Uracil
  modified_base 38: mod_base = OTHER; note = Uracil
  modified_base 39: mod_base = OTHER; note = Uracil
  modified_base 44: mod_base = OTHER; note = Uracil

SEQ ID NO: 25 - RNA, 48 nt, synthetic construct
  misc_feature 1..48: note = Synthetic
  misc_feature 24: note = propyl linker
  source 1..48: mol_type = other RNA
aagatgaacttcagggtcagcgtcaagatgaacttcagggtcagcgtc

SEQ ID NO: 26 - DNA, 48 bp, synthetic construct; identical to SEQ ID NO: 6
  misc_feature 1..48: note = Synthetic
  misc_feature 1..24: note = 3'-5' orientation
  misc_feature 24: note = triethylene glycol linker
  source 1..48: mol_type = other DNA
  modified_base 1: mod_base = OTHER; note = Uracil
  modified_base 2: mod_base = OTHER; note = Uracil
  modified_base 4: mod_base = OTHER; note = Uracil
  modified_base 7: mod_base = OTHER; note = Uracil
  modified_base 8: mod_base = OTHER; note = Uracil
  modified_base 13: mod_base = OTHER; note = Uracil
  modified_base 19: mod_base = OTHER; note = Uracil
  modified_base 30: mod_base = OTHER; note = Uracil
  modified_base 36: mod_base = OTHER; note = Uracil
  modified_base 41: mod_base = OTHER; note = Uracil
  modified_base 42: mod_base = OTHER; note = Uracil
  modified_base 45: mod_base = OTHER; note = Uracil
  modified_base 47: mod_base = OTHER; note = Uracil
  modified_base 48: mod_base = OTHER; note = Uracil